(19) United States
(12) Patent Application Publication
(10) Pub. No.: US 2024/0052103 A1
(43) Pub. Date: Feb. 15, 2024

(54) POLYMER AND TRIAZINE COMPOUND

(71) Applicant: TOKYO OHKA KOGYO CO., LTD., Kawasaki-shi (JP)

(72) Inventors: Takahiro Shimizu, Kawasaki-shi (JP); Shigeo Yasuda, Kawasaki-shi (JP); Shingo Isobe, Kawasaki-shi (JP)

(21) Appl. No.: 18/355,899

(22) Filed: Jul. 20, 2023

(30) Foreign Application Priority Data
Aug. 2, 2022 (JP) 2022-123504

(51) Int. Cl. C08G 73/06; C07D 251/42; C08J 5/18

(52) U.S. Cl. CPC C08G 73/0644; C08G 73/065; C07D 251/42; C08J 5/18; G02B 1/041

(57) ABSTRACT

A polymer having a high refractive index and providing a molded product that has excellent solvent resistance; a triazine compound; a method for producing the triazine compound; a method for producing the polymer using the triazine compound; a non-photosensitive liquid composition including the polymer; a method for producing a molded product using the composition; a molded product of the composition; a film formed from the molded product; a microlens formed from the molded product; and an optical element including the microlens. A polymer including a unit having a specific structure in which an aromatic group having a hydroxy group protected with a tert-butoxycarbonyloxy group, a tert-butoxycarbonylamino group, or an acetal protecting group is linked to a triazine ring through a specific linking group.

### RELATED APPLICATION

This application claims priority to Japanese Patent Application No. 2022-123504, filed Aug. 2, 2022, the entire content of which is incorporated herein by reference.

### BACKGROUND OF THE INVENTION

### Field of the Invention

The present invention relates to a polymer including a unit having a specific structure; a triazine compound capable of being suitably used as a raw material of the polymer; a method for producing the triazine compound; a method for producing the above-described polymer using the triazine compound; a non-photosensitive liquid composition including the above-described polymer; a method for producing a molded product using the non-photosensitive liquid composition; a molded product of the above-described non-photosensitive liquid composition; a film formed of the above-described molded product; a microlens formed of the above-described molded product; and an optical element including the microlens.

### Related Art

Conventionally, a solid-state image sensor has been used for a camera, a video camera, and the like. A charge-coupled device (CCD) image sensor or a complementary metal-oxide semiconductor (CMOS) image sensor has been used as the solid-state image sensor. The image sensor includes a fine condensing lens for the purpose of improving condensing efficiency (hereinafter referred to as microlens).

Recently, the CCD image sensor and the CMOS image sensor have had a higher resolution. Therefore, the microlens is becoming smaller in diameter. A material of the image sensor needs to have a higher refractive index in order to condense light to a photodiode in the image sensor with high efficiency even though the microlens has a small diameter. For example, a linear polymer including a triazine ring has been known as a highly-refractive material capable of being molded into various forms (see, Patent Document 1).

Patent Document 1: Japanese Unexamined Patent Application, Publication No. 2014-162829

### SUMMARY OF THE INVENTION

Use of the linear polymer described in Patent Document 1 enables formation of a highly-refractive microlens. However, a sensor including a microlens is often exposed to a liquid such as an organic solvent during production of a device including the sensor. Therefore, a material of the microlens needs to have excellent solvent resistance.

The present invention is proposed in view of such circumstances, and an object thereof is to provide a polymer having a high refractive index and providing a molded product that has excellent solvent resistance; a triazine compound capable of being suitably used as a raw material of the polymer; a method for producing the triazine compound; a method for producing the above-described polymer using the triazine compound; a non-photosensitive liquid composition including the above-described polymer; a method for producing a molded product using the non-photosensitive liquid composition; a molded product of the above-described non-photosensitive liquid composition; a film formed of the above-described molded product; a microlens formed of the above-described molded product; and an optical element including the microlens.

The present inventors have found that the above-mentioned problem can be solved by using a polymer including a unit having a specific structure in which an aromatic group having a hydroxy group protected with a tert-butoxycarbonyloxy group, a tert-butoxycarbonylamino group, or an acetal protecting group is linked to a triazine ring through a specific linking group. Thus, the present invention has been completed. Specifically, the present invention provides the following aspects.

A first aspect of the present invention relates to a polymer including a unit represented by Formula (A1) below:

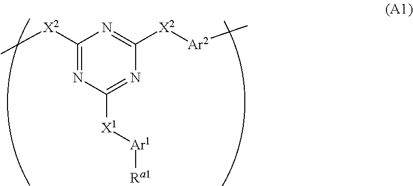

in which Ar¹ and Ar² are an aromatic group-containing group, R^{a1} is a hydroxy group protected with a tert-butoxycarbonyloxy group, a tert-butoxycarbonylamino group, or an acetal protecting group, the acetal protecting group being a group represented by —CHR^{A1}—O—R^{A2}, R^{A1} is a hydrogen atom or an alkyl group, R^{A2} is an alkyl group, R^{A1} and R^{A2} may be linked to each other to form a ring, X¹ and X² are each independently —NR^{a2}—, —O—, or —S—, R^{A2} is a hydrogen atom, an optionally substituted alkyl group, or an optionally substituted aromatic hydrocarbon group, X¹ is linked to an aromatic ring in an aromatic group-containing group serving as Ar¹, and X² is linked to an aromatic ring in an aromatic group-containing group serving as Ar².

A second aspect of the present invention relates to a triazine compound represented by Formula (A3) below:

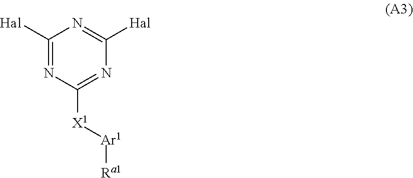

in which Hal is a halogen atom, Ar¹ is an aromatic group-containing group, R^{a1} is a hydroxy group protected with a tert-butoxycarbonyloxy group, a tert-butoxycarbonylamino group, or an acetal protecting group, the acetal protecting group being a group represented by —CHR^{A1}—O—R^{A2}, R^{A1} is a hydrogen atom or an alkyl group, R^{A2} is an alkyl group, R^{A1} and R^{A2} may be linked to each other to form a ring, X¹ is —NR^{a2}—, —O—, or —S—, R^{A2} is a hydrogen atom, an optionally substituted alkyl group, or an optionally substituted aromatic hydrocarbon group, and X¹ is linked to an aromatic ring in an aromatic group-containing group serving as Ar¹.

A third aspect of the present invention relates to a method for producing the triazine compound according to the second aspect, the method including condensing cyanuric halide with a compound represented by any of Formulae (A3-1) to (A3-3) below:

R^{a1}—Ar¹—NR^{a2}H (A3-1)

R^{a1}—Ar¹—OH (A3-2)

R^{a1}—Ar¹—SH (A3-3)

in which Ar¹ is an aromatic group-containing group, R^{a1} is a hydroxy group protected with a tert-butoxycarbonyloxy group, a tert-butoxycarbonylamino group, or an acetal protecting group, and R^{a2} is a hydrogen atom, an optionally substituted alkyl group, or an optionally substituted aromatic hydrocarbon group.

A fourth aspect of the present invention relates to a method for producing a polymer, the method including condensing the triazine compound according to the second aspect to a compound represented by any of Formulae (A4-1) to (A4-3) below:

Ar²—(NR^{a2} H)² (A4-1)

Ar²—(OH)² (A4-2)

Ar²—(SH)² (A4-3)

in which Ar² is an aromatic group-containing group, and R^{a2} is a hydrogen atom, an optionally substituted alkyl group, or an optionally substituted aromatic hydrocarbon group.

A fifth aspect of the present invention relates to a non-photosensitive liquid composition including:
a polymer (A) according to the first aspect; and
an organic solvent (S), and
further including a thermal acid generating agent (C) when R^{a1} is a hydroxy group protected with an acetal protecting group.

A sixth aspect of the present invention relates to a method for forming a molded product, the method including: molding the non-photosensitive liquid composition according to the fifth aspect; and
removing the organic solvent (S) with heating from the non-photosensitive liquid composition having been molded.

A seventh aspect of the present invention relates to a molded product of the non-photosensitive liquid composition according to the fifth aspect.

An eighth aspect of the present invention relates to a film formed of a molded product according to the seventh aspect.

A ninth aspect of the present invention relates to a microlens formed of the molded product according to the seventh aspect.

A tenth aspect of the present invention relates to an optical element including the microlens according to the ninth aspect.

The present invention can provide a polymer having a high refractive index and providing a molded product that has excellent solvent resistance; a triazine compound capable of being suitably used as a raw material of the polymer; a method for producing the triazine compound; a method for producing the above-described polymer using the triazine compound; a non-photosensitive liquid composition including the above-described polymer; a method for producing a molded product using the non-photosensitive liquid composition; a molded product of the above-described non-photosensitive liquid composition; a film formed of the above-described molded product; a microlens formed of the above-described molded product; and an optical element including the microlens.

### DETAILED DESCRIPTION OF THE INVENTION

### <<Polymer>>

A polymer includes a unit represented by Formula (A1) below. The polymer including a unit represented by Formula (A1) exhibits a high refractive index.

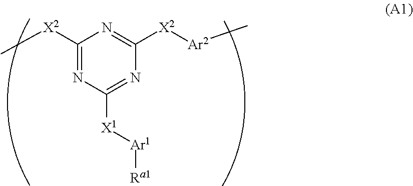

In Formula (A1), Ar¹ and Ar² are an aromatic group-containing group. R^{a1} is a hydroxy group protected with a tert-butoxycarbonyloxy group, a tert-butoxycarbonylamino group, or an acetal protecting group, the acetal protecting group being a group represented by —CHR^{A1}—O—R^{A2}. R^{A1} is a hydrogen atom or an alkyl group. R^{A2} is an alkyl group. R^{A1} and R^{A2} may be linked to each other to form a ring. X¹ and X² are each independently —NR^{a2}—, —O—, or —S—. R^{a2} is a hydrogen atom, an optionally substituted alkyl group, or an optionally substituted aromatic hydrocarbon group. X¹ is linked to an aromatic ring in an aromatic group-containing group serving as Ar¹. X² is linked to an aromatic ring in an aromatic group-containing group serving as Ar².

The polymer including a unit represented by Formula (A1) has a hydroxy group protected with a tert-butoxycarbonyloxy group, a tert-butoxycarbonylamino group, or an acetal protecting group. Such a polymer is soluble in various organic solvents such as a nitrogen-containing polar organic solvent and a ketone solvent. However, deprotection of the hydroxy group protected with a tert-butoxycarbonyloxy group, a tert-butoxycarbonylamino group, or an acetal protecting group makes the polymer less soluble in the organic solvent. Cross-linking of a deprotected polymer having a hydroxy group or an amino group with a polymerizable monomer capable of reacting with the hydroxy group or the amino group makes the polymer much less soluble in the organic solvent.

Therefore, the above-described polymer is easily processed as a solution. On the other hand, a molded product produced using the above-described polymer under a condition under which the hydroxy group protected with a tert-butoxycarbonyloxy group, a tert-butoxycarbonylamino group, or an acetal protecting group is deprotected is less soluble in the organic solvent.

In addition to the unit represented by Formula (A1), the polymer may include another unit other than the unit represented by Formula (A1). The unit represented by Formula (A1) is hereinafter also referred to as “unit (A1)”. The other unit other than the unit represented by Formula (A1) is hereinafter also referred to as “unit (A2)”. An amount of the unit (A1) in the polymer is not particularly limited unless a desired effect is impaired. The amount of the unit (A1) in the polymer is preferably 50% by mass or more, more preferably 70% by mass or more, further preferably 80% by mass or more, particularly preferably 90% by mass or more, and most preferably 100% by mass relative to a mass of the polymer.

### <Unit (A1)>

In Formula (A1), Ar¹ and Ar² are an aromatic group-containing group. The aromatic group-containing group may be composed of only an aromatic group or may have an aromatic group and a non-aromatic group. The aromatic group-containing group may include only one aromatic group or two or more aromatic groups. The aromatic group included in the aromatic group-containing group may be an aromatic hydrocarbon group or an aromatic heterocyclic group. An aromatic group included in the aromatic group-containing group is preferably an aromatic hydrocarbon group.

The aromatic group included in the aromatic group-containing group may be a monocyclic group or a polycyclic group. The polycyclic group may be a fused cyclic group or a group in which one or more rings selected from a monocyclic group and a polycyclic group are linked by a single bond. The fused cyclic group may be a group in which aromatic groups are fused with each other or a group in which an aromatic group is fused with an aliphatic cyclic group.

A number of an aromatic monocycle included in the aromatic group-containing group is not particularly limited unless a desired effect is impaired. Note that, when the aromatic group-containing group is an aromatic fused ring group, a number of a monocycle constituting the aromatic fused ring shall be the number of the aromatic monocycle included in the aromatic group-containing group. Specifically, when the aromatic group-containing group is a naphthalenediyl group, the number of the aromatic monocycle included in the aromatic group-containing group is 2. The number of the aromatic monocycle included in the aromatic group-containing group is preferably 1 or more and 4 or less, more preferably 1 or more and 3 or less, and further preferably 1 or 2.

Suitable examples of the aromatic group-containing group include groups represented by Formula (a-1) to Formula (a-11) below.

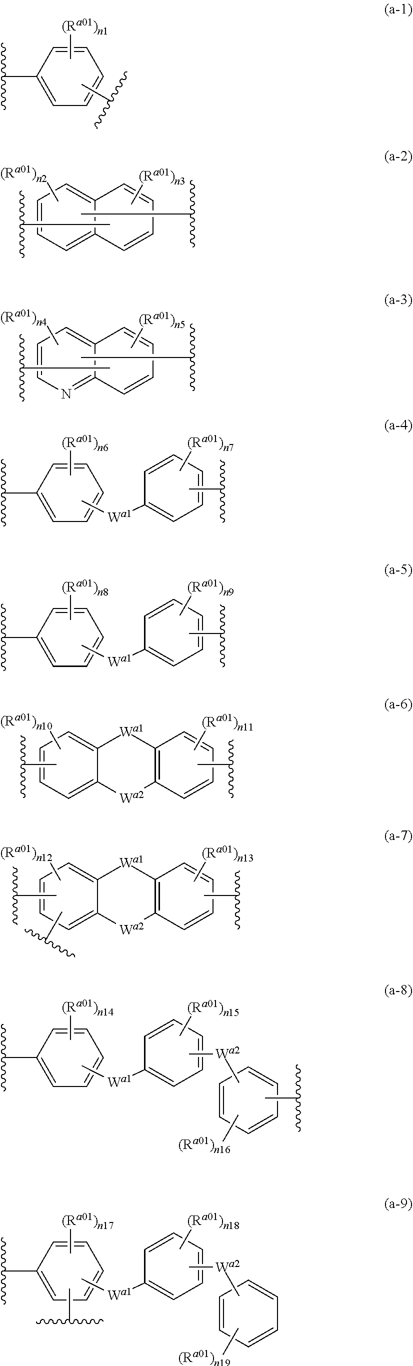

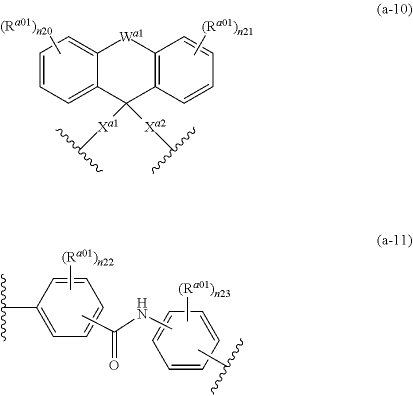

In Formula (a-1) to Formula (a-11), R^{a01} is each independently a group selected from the group consisting of a halogen atom, a sulfonate group, an alkyl group having 1 or more and 10 or less carbon atoms, and an alkoxy group having 1 or more and 10 or less carbon atoms. R^{a02} is a hydrogen atom, an alkyl group having 1 or more and 10 or less carbon atoms, an aromatic hydrocarbon group having 6 or more and 12 or less carbon atoms, or an aralkyl group having 7 or more and 13 or less carbon atoms. W^{a1} and W^{a2} are each independently a single bond, a group represented by —CR^{a03}R^{a04}—, a carbonyl group, or a group represented by —O—, —S—, —SO—, —SO₂—, or —NR^{a05}—. R^{a03} and R^{a04} are each a hydrogen atom or an alkyl group having 1 or more and 10 or less carbon atoms. In the group represented by —CR^{a03}R^{a04}—, R^{a03} and R^{a04} may be linked to each other to form a ring. R^{a05} is a hydrogen atom or an alkyl group having 1 or more and 10 or less carbon atoms. X^{a1} and X^{a2} are each independently a single bond, an alkylene group having 1 or more and 10 or less carbon atoms, or a group represented by —Y^{a1}—Ph—Y^{a2}—. Ph is a phenylene group optionally having 1 or more and 4 or less substituents. The substituent that the phenylene group optionally has is a group selected from the group consisting of a halogen atom, a sulfonate group, an alkyl group having 1 or more and 10 or less carbon atoms, and an alkoxy group having 1 or more and 10 or less carbon atoms. Y^{a1} and Y^{a2} are each independently a single bond or an alkylene group having 1 or more and 10 or less carbon atoms. n1 is an integer of 0 or more and 4 or less. n2 and n3 are each independently an integer of 0 or more and 3 or less. n4 is an integer of 0 or more and 2 or less. n5 is an integer of 0 or more and 3 or less. n6 and n7 are each independently an integer of 0 or more and 3 or less. n8 is an integer of 0 or more and 3 or less. n9 is an integer of 0 or more and 5 or less. n10 and n11 are each independently an integer of 0 or more and 3 or less. n12 is an integer of 0 or more and 3 or less. n13 is an integer of 0 or more and 4 or less. n14, n15, and n16 are each independently an integer of 0 or more and 4 or less. n17 is an integer of 0 or more and 3 or less. n18 is an integer of 0 or more and 4 or less. n19 is an integer of 0 or more and 5 or less. n20 and n21 are an integer of 0 or more and 4 or less. n22 and n23 are an integer of 0 or more and 4 or less.

Examples of the halogen atom serving as Ram include a fluorine atom, a chlorine atom, a bromine atom, and an iodine atom. Among them, the fluorine atom, the chlorine atom, and the bromine atom are preferred.

The alkyl group having 1 or more and 10 or less carbon atoms serving as R^{a01} may be linear or branched. Specific examples of the alkyl group having 1 or more and 10 or less carbon atoms serving as R^{a01} include a methyl group, an ethyl group, an n-propyl group, an isopropyl group, an n-butyl group, an isobutyl group, a sec-butyl group, a tert-butyl group, an n-pentyl group, an isopentyl group, a tert-pentyl group, an n-hexyl group, an n-heptyl group, an n-octyl group, a 2-ethyl-n-hexyl group, an n-nonyl group, and an n-decyl group.

The alkoxy group having 1 or more and 10 or less carbon atoms serving as R^{a01} may be linear or branched. Specific examples of the alkoxy group having 1 or more and 10 or less carbon atoms serving as R^{a01} include a methoxy group, an ethoxy group, an n-propyloxy group, an isopropyloxy group, an n-butyloxy group, an isobutyloxy group, a sec-butyloxy group, a tert-butyloxy group, an n-pentyloxy group, an isopentyloxy group, a tert-pentyloxy group, an n-hexyloxy group, an n-heptyloxy group, an n-octyloxy group, a 2-ethyl-n-hexyloxy group, an n-nonyloxy group, and an n-decyloxy group.

The alkyl group having 1 or more and 10 or less carbon atoms serving as R^{a02} is the same as described for the alkyl group having 1 or more and 10 or less carbon atoms serving as R^{a01}.

Specific examples of the aromatic hydrocarbon group having 6 or more 12 or less carbon atoms serving as R^{a02} include a phenyl group, a naphthalene-1-yl group, a naphthalene-2-yl group, a 4-phenylphenyl group, a 3-phenylphenyl group, and a 2-phenylphenyl group.

Specific examples of the aralkyl group having 7 or more and 13 or less carbon atoms include a benzyl group, a phenethyl group, a 3-phenyl-n-propyl group, a phthalene-1-ylmethyl group, and a phthalene-2-ylmethyl group.

W^{a1} and W^{a2} are each independently a single bond, a group represented by —CR^{a03}R^{a04}—, a carbonyl group, or a group represented by —O—, —S—, —SO—, —SO₂—, or —NR^{a05}—. When W^{a1} and W^{a2} are each the group represented by —CR^{a03}R^{a04}—, R^{a03} and R^{a04} are each a hydrogen atom or an alkyl group having 1 or more and 10 or less carbon atoms. The alkyl group having 1 or more and 10 or less carbon atoms serving as R^{a03} and R^{a04} is the same as described for the alkyl group having 1 or more and 10 or less carbon atoms serving as R^{a01}. R^{a03} and R^{a04} may be linked to each other to form a ring. When W^{a1} and W^{a2} are each the group represented by —NR^{a05}—, R^{a05} is a hydrogen atom or an alkyl group having 1 or more and 10 or less carbon atoms. The alkyl group having 1 or more and 10 or less carbon atoms serving as R^{a05} is the same as described for the alkyl group having 1 or more and 10 or less carbon atoms serving as R^{a01}.

X^{a1} and X^{a2} are each independently a single bond, an alkylene group having 1 or more and 10 or less carbon atoms, or a group represented by —Y^{a1}—Ph—Y^{a2}—. Specific examples of the alkylene having 1 or more and 10 or less carbon atoms serving as X^{a1} and X^{a2} include a methylene group, an ethane-1,2-diyl group (ethylene group), an ethane-1,1-diyl group, a propane-1,3-diyl group, a propane-1,2-diyl group, a propane-1,1-diyl group, a propane-2,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, and a decane-1,10-yl group. When X^{a1} and X^{a2} are each the group represented by —Y^{a1}—Ph—Y^{a2}—, Ph is a phenylene group optionally having 1 or more and 4 or less substituents. The substituent that the phenylene group optionally has is a group selected from the group consisting of a halogen atom, a sulfonate group, an alkyl group having 1 or more and 10 or less carbon atoms, and an alkoxy group having 1 or more and 10 or less carbon atoms. The alkyl group having 1 or more and 10 or less carbon atoms and the alkoxy group having 1 or more and 10 or less carbon atoms serving as the substituent that the phenylene group optionally has are the same as described for the alkyl group having 1 or more and 10 or less carbon atoms and the alkoxy group having 1 or more and 10 or less carbon atoms serving as R^{a01}. Y^{a1} and Y^{a2} are each independently a single bond or an alkylene group having 1 or more and 10 or less carbon atoms. The alkylene group having 1 or more and 10 or less carbon atoms serving as Y^{a1} and Y^{a2} is the same as described for the alkylene group having 1 or more and 10 or less carbon atoms serving as X^{a1} and X^{a2}.

Specific examples of the group represented by any of Formulae (a-1) to (a-11) include the following groups.

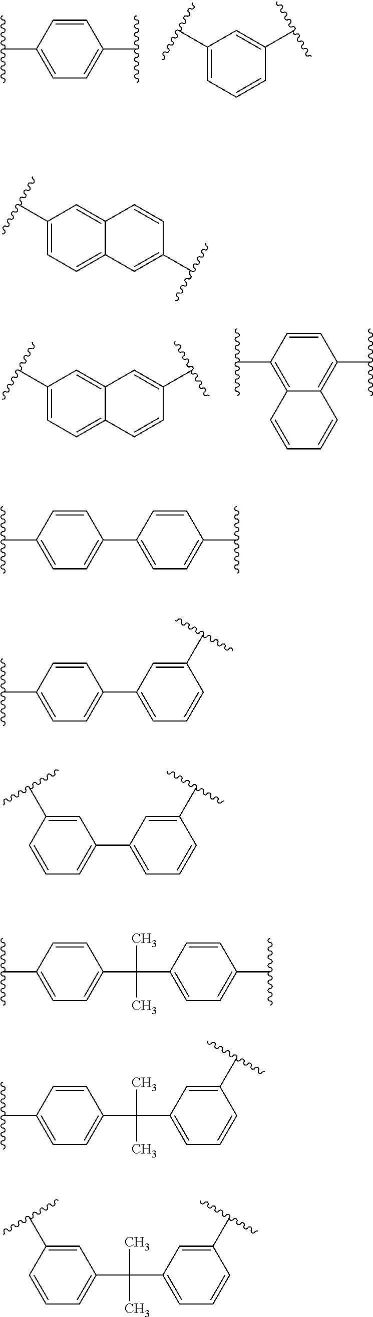

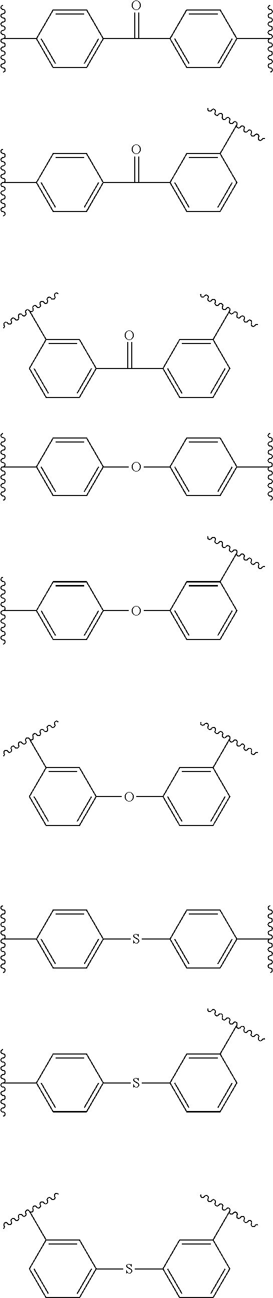

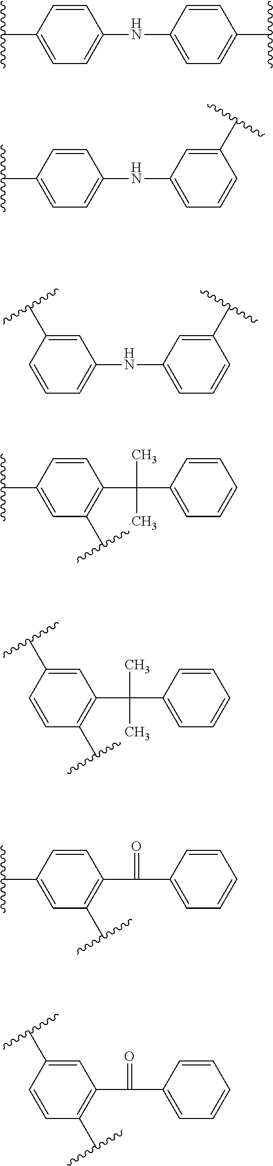

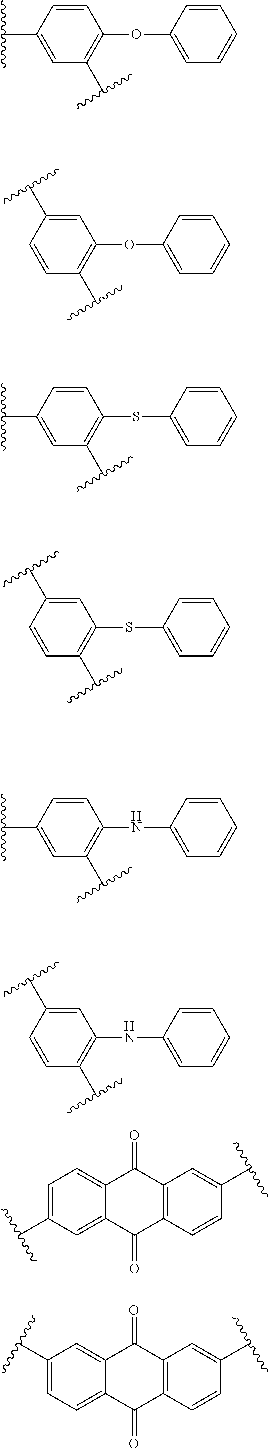

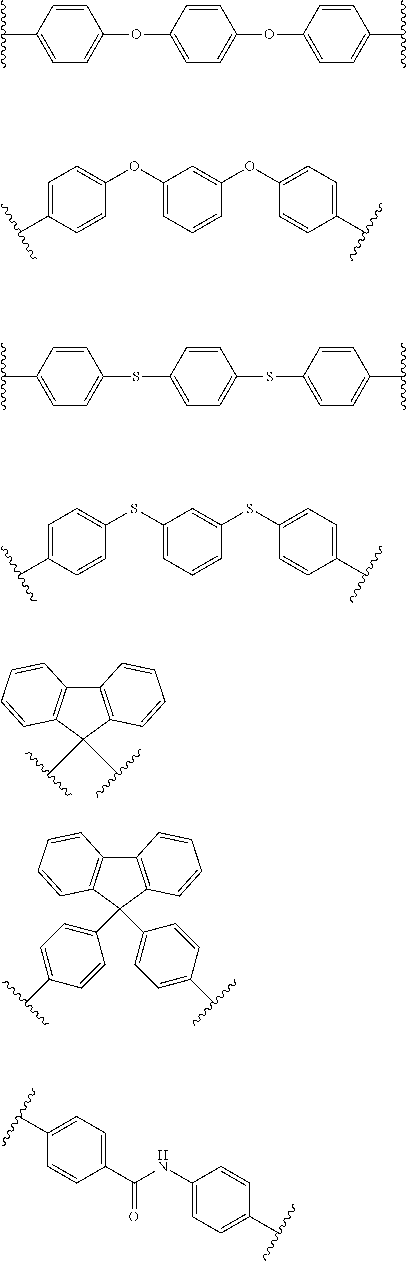

Among the groups above, the following groups are preferred.

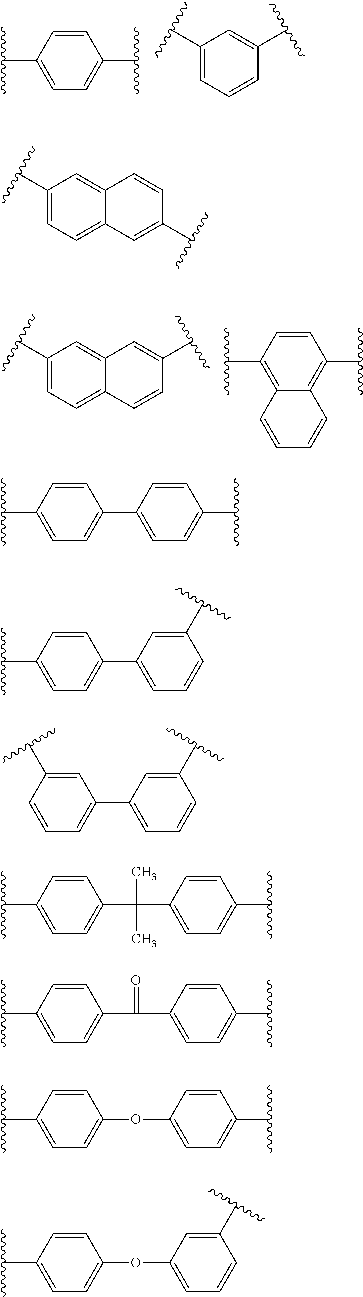

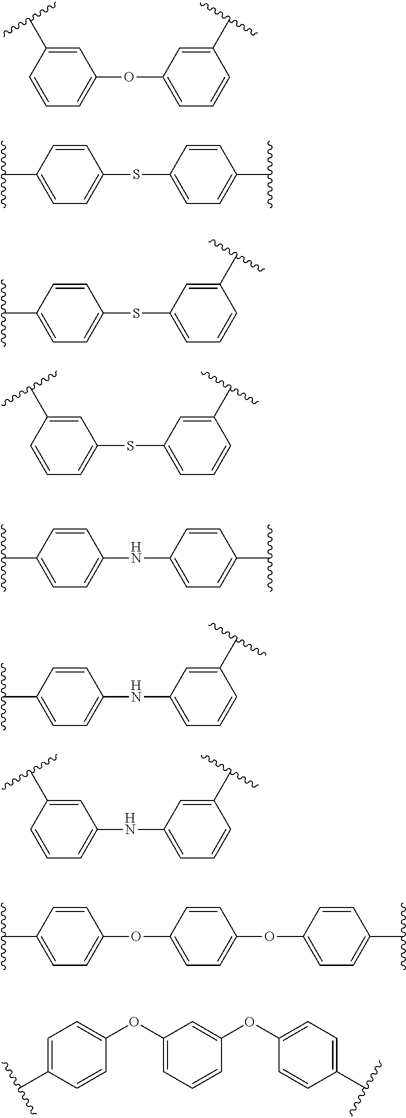

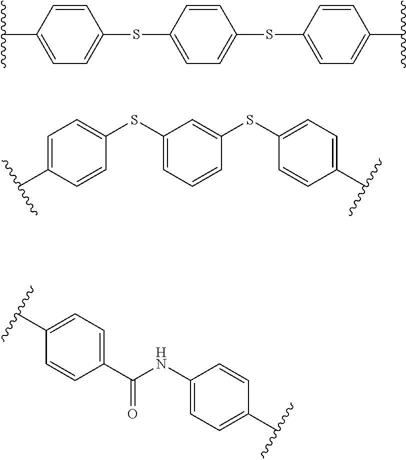

In Formula (A1), R^{a1} is a hydroxy group protected with a tert-butoxycarbonyloxy group, a tert-butoxycarbonylamino group, or an acetal protecting group. The tert-butoxycarbonyloxy group and the tert-butoxycarbonylamino group can be deprotected with only heating. On the other hand, the hydroxy group protected with an acetal protecting group is difficult to deprotect with only heating. In order to deprotect the hydroxy group protected with an acetal protecting group, for example, the protected hydroxy group is advantageously heated in the coexistence of a thermal acid generating agent (C) described below. In this case, the hydroxy group protected with an acetal protecting group is easily deprotected by the action of an acid that the thermal acid generating agent (C) generates upon heating. As described above, from the viewpoint of easy deprotection with only heating, R^{a1} is preferably a tert-butoxycarbonyloxy group or a tert-butoxycarbonylamino group and more preferably a tert-butoxycarbonyloxy group.

The acetal protecting group is a group represented by —CHR^{A1}—O—R^{A2}. R^{A1} is a hydrogen atom or an alkyl group. R^{A2} is an alkyl group. R^{A1} and R^{A2} may be linked to each other to form a ring. A number of carbon atoms in the alkyl group serving as R^{A1} and R^{A2} is preferably 1 or more and 6 or less and more preferably 1 or more and 4 or less. The alkyl group serving as R^{A1} and R^{A2} may be linear or branched, but is preferably linear. Suitable specific examples of the alkyl group serving as R^{A1} and R^{A2} include a methyl group, an ethyl group, an n-propyl group, and an n-butyl group.

Specific examples of the group represented by —CHR^{A1}—O—R^{A2} include a methoxymethyl group, an ethoxymethyl group, an n-propyloxymethyl group, an n-butyloxymethyl group, a 1-methoxyethyl group, a 1-ethoxymethyl group, a 1-n-propyloxyethyl group, a 1-n-butyloxyethyl group, a tetrahydropyran-2-yl group, and a tetrahydrofuran-2-yl group.

In Formula(A1), X¹ and X² are each independently —NR^{a2}—, —O—, or —S—. R^{A2} is a hydrogen atom, an optionally substituted alkyl group, or an optionally substituted aromatic hydrocarbon group.

A number of carbon atoms in the alkyl group serving as Ra 2 is not particularly limited. A number of carbon atoms in the alkyl group serving as R^{a2} is preferably 1 or more and 6 or less and more preferably 1 or more and 4 or less. The alkyl group serving as R^{a2} may be linear or branched, but is preferably linear. Specific examples of the alkyl group serving as R^{a2} include a methyl group, an ethyl group, an n-propyl group, an isopropyl group, an n-butyl group, an isobutyl group, a sec-butyl group, a tert-butyl group, an n-pentyl group, and n-hexyl group. Examples of the substituent that the alkyl group serving as R^{a2} optionally has include a halogen atom, a hydroxy group, an alkoxy group, a mercapto group, and a cyano group.

A number of carbon atoms in the aromatic hydrocarbon group serving as R^{a2} is not particularly limited. A number of carbon atoms in the alkyl group serving as R^{a2} is preferably 6 or more and 12 or less. Specific examples of the aromatic hydrocarbon group serving as R^{a2} include a phenyl group, a naphthalene-1-yl group, a naphthalene-2-yl group, a 4-phenylphenyl group, a 3-phenylphenyl group, and a 2-phenylphenyl group. Examples of the substituent that the aromatic hydrocarbon group serving as R^{a2} optionally has include a halogen atom, an alkyl group, a hydroxy group, an alkoxy group, a mercapto group, and a cyano group.

X¹ in Formula (A1) is linked to an aromatic ring in an aromatic group-containing group serving as Ar¹. X² in Formula (A1) is linked to an aromatic ring in an aromatic group-containing group serving as Ar².

The above-mentioned X² is preferably —NR^{a2}— or —O— and more preferably —NR^{a2}—. When X² is —NR^{a2}—, a torsion between a triazine ring and Ar² is small in a unit represented by Formula (A1). As a result, the unit represented by Formula (A1) has high planarity. It is believed that when the unit represented by Formula (A1) has high planarity, a polymer having the unit represented by Formula (A1) has a high refractive index.

Preferred specific examples of the unit (A1) include the below-mentioned units. A unit in which an amino group to be linked to a triazine ring in any of the below-mentioned units is changed to —O— or —S— is also preferred. A unit in which a tert-butoxycarbonyloxy group in any of the following units is changed to a tert-butoxycarbonylamino group is also preferred. A unit in which a tert-butoxycarbonyl group in any of the below-mentioned units is changed to a methoxymethyl group, an ethoxymethyl group, an n-propyloxymethyl group, an n-butyloxymethyl group, a 1-methoxyethyl group, a 1-ethoxymethyl group, a 1-n-propyloxyethyl group, a 1-n-butyloxyethyl group, a tetrahydropyran-2-yl group, or a tetrahydrofuran-2-yl group is also preferred.

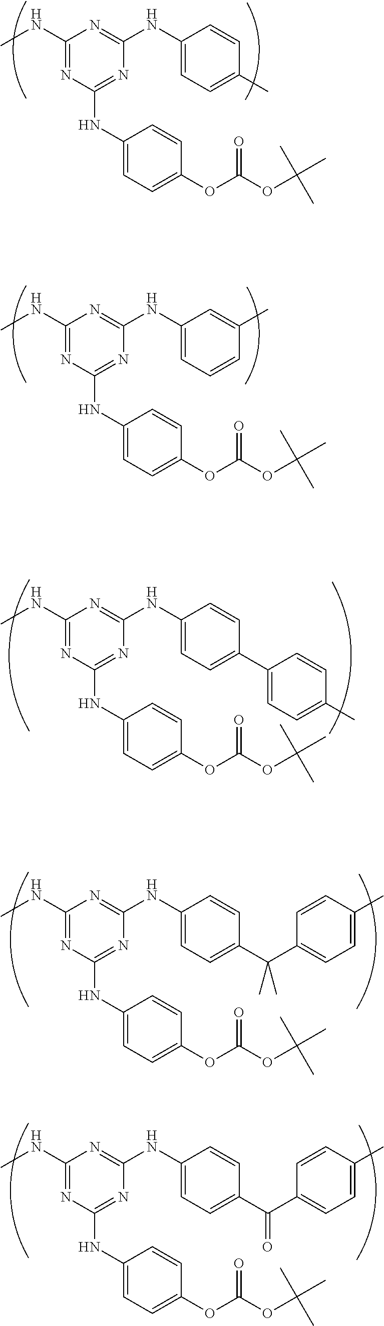

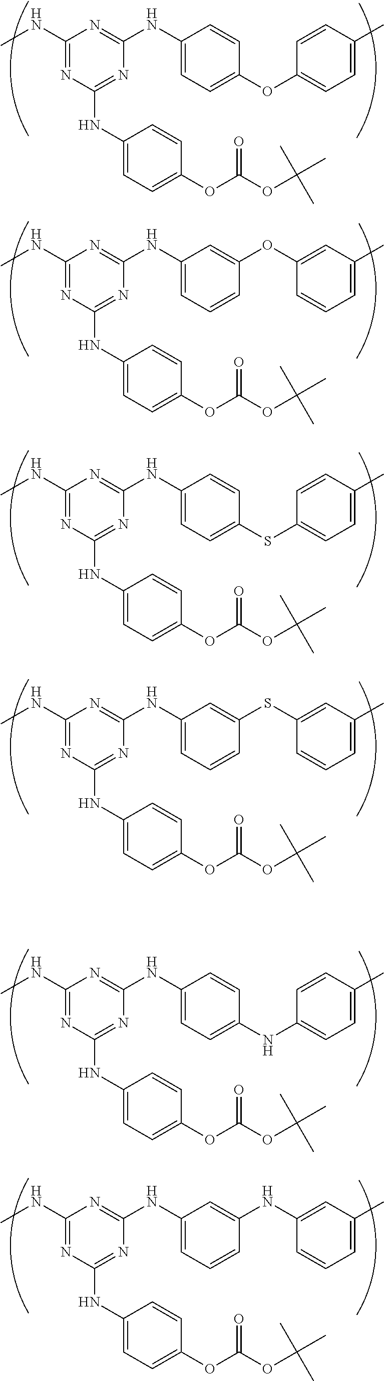

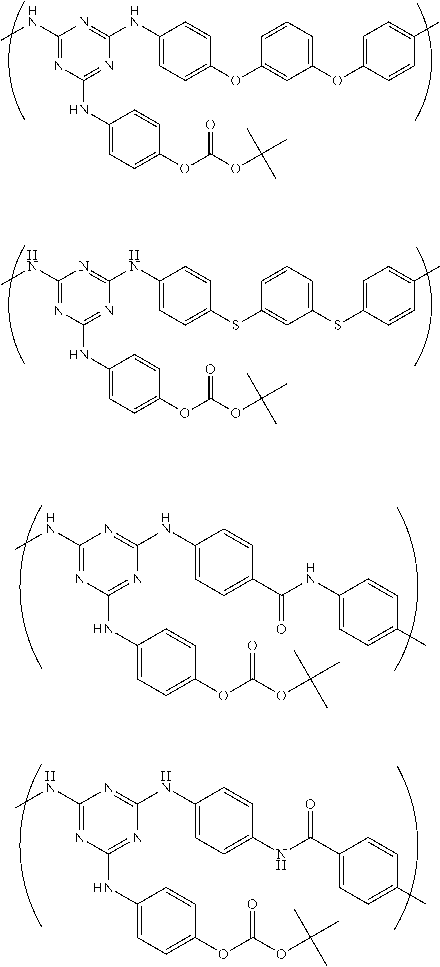

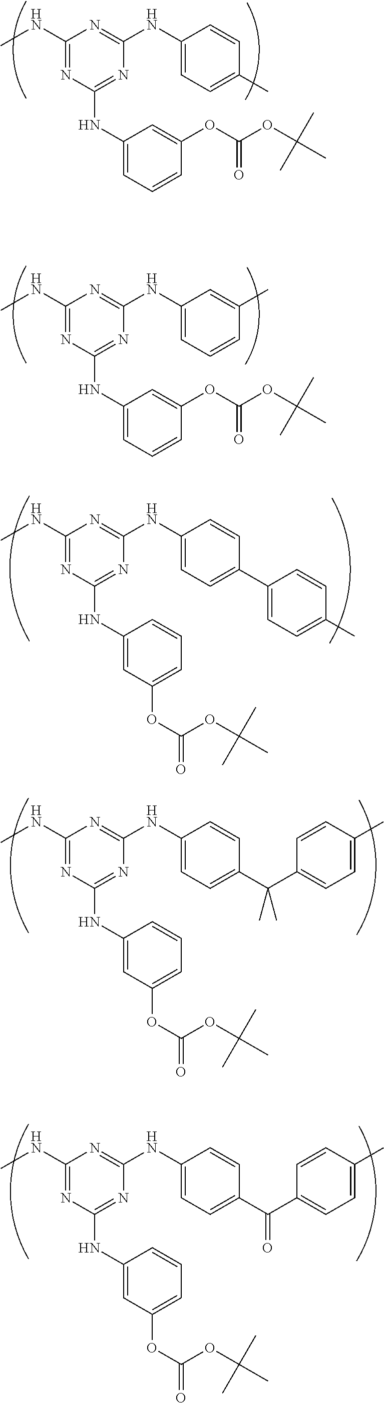

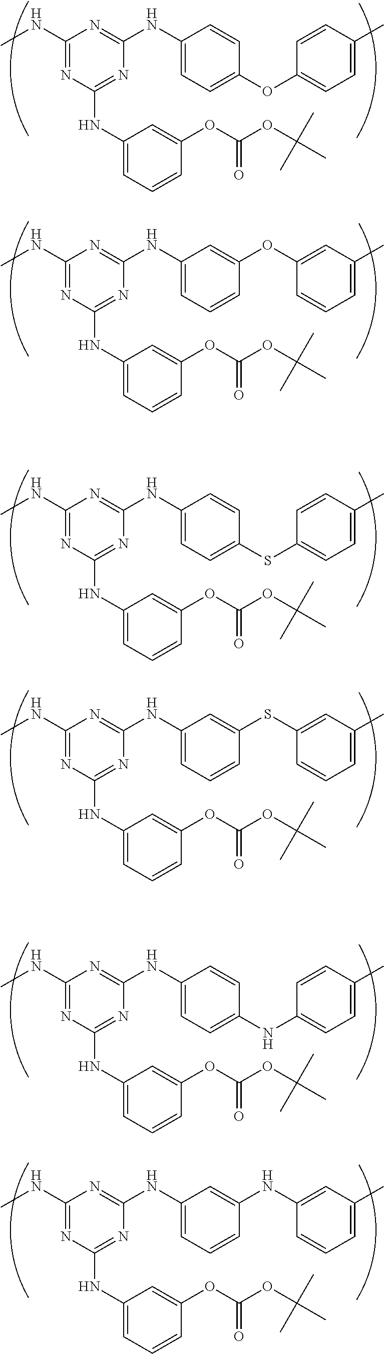

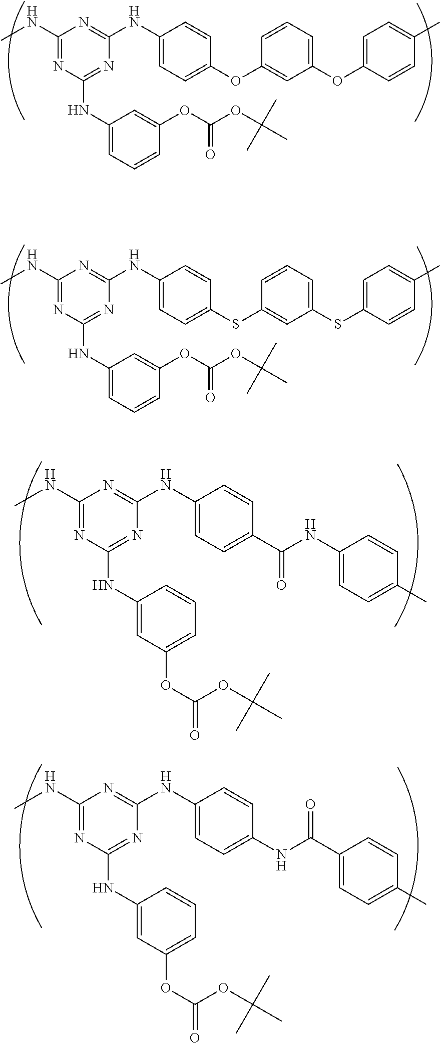

### <Unit (A2)>

As mentioned above, the polymer may include a unit (A2) that is another unit other than the unit represented by Formula (A1). A structure of the unit (A2) is not particularly limited unless a desired effect is impaired.

The polymer is produced by polymerizing a dihalotriazine compound with an aromatic diamine compound, an aromatic diol, or an aromatic dithiol, as mentioned below. Therefore, a unit represented by Formula (A2) below is preferred as the unit (A2).

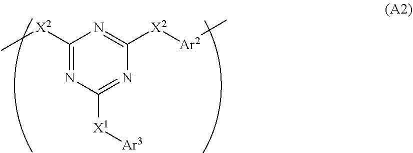

In Formula (A2), X¹, X², and Ar² are the same as described for Formula (A1). In Formula (A2), Ar³ is an optionally substituted aromatic group. The aromatic group serving as Ar³ may be an aromatic hydrocarbon group or an aromatic heterocyclic group. The aromatic group serving as Ar³ is preferably an aromatic hydrocarbon group. Specific examples of the aromatic hydrocarbon group serving as Ar³ include a phenyl group, a naphthalene-1-yl group, a naphthalene-2-yl group, a 4-phenylphenyl group, a 3-phenylphenyl group, and a 2-phenylphenyl group. Examples of the substituent that the aromatic hydrocarbon group serving as Ar³ optionally has include a halogen atom, an alkyl group, an alkoxy group, and a cyano group.

A weight average molecular weight of the polymer is not particularly limited unless a desired effect is impaired. The weight average molecular weight is preferably 500 or more and 100000 or less. The weight average molecular weight is preferably 5000 or more from the viewpoint of a high solvent resistance of the polymer under heating. The weight average molecular weight is preferably 30000 or less from the viewpoint of a high solubility of the polymer in various solvents.

### <Method for Producing Polymer>

A method for producing the above-mentioned polymer is not particularly limited as long as the resulting polymer includes the unit (A1). Typically, the polymer is produced by condensing a triazine compound represented by Formula (A3) below with an aromatic diamine compound represented by Formula (A5-1) below, an aromatic diol represented by Formula (A5-2) below, or an aromatic dithiol represented by Formula (A5-3).

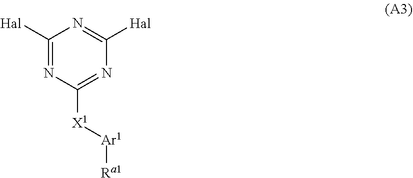

In Formula (A3), Ar¹, X¹, and R^{a1} are the same as described for Formula (A1). Hal is a halogen atom. Examples of Hal include a fluorine atom, a chlorine atom, a bromine atom, and an iodine atom. Among them, the chlorine atom and bromine atom are preferred and the chlorine atom is more preferred.

Ar²—(NR^{a2}H)₂ (A5-1)

Ar²—(OH)₂ (A5-2)

Ar²—(SH)₂ (A5-3)

In Formula (A5-1) to Formula (A5-3), Are and Rae are the same as described for Formula (A1).

Specific examples of the aromatic diamine compound represented by Formula (A5-1) include p-phenylenediamine, m-phenylenediamine, 2,4-diaminotoluene, 4,4′-diaminobiphenyl, 3,3′-diaminobiphenyl, 3,4′-diaminobiphenyl, 1,4-diaminonaphthalene, 1,5-diaminonaphthalene, 2,6-diaminonaphthalene, 2,7-diaminonaphthalene, 9,10-diaminoanthracene, 9,10-bis(4-aminophenyl)anthracene, 4,4′-diamino-2,2′-bis(trifluoromethyl)biphenyl, 4,4′-diaminobenzophenone, 3,3′-diaminobenzophenone, 3,4′-diaminobenzophenone, 4,4′-diaminodiphenylsulfone, 3,3′-diaminodiphenylsulfone, 3,4′-diaminodiphenylsulfone, 4,4′-diaminodiphenylsulfide, 3,3′-diaminodiphenylsulfide, 3,4′-diaminodiphenylsulfide, 4,4′-diaminodiphenylmethane, 3,3′-diaminodiphenylmethane, 3,4′-diaminodiphenylmethane, 2,2-bis(4-aminophenyl)propane, 2,2-bis[4-(4-aminophenoxy)phenyl]propane, 2,2-bis[4-(4-aminophenoxy)phenyl]hexafluoropropane, 4,4′-diaminodiphenylether, 3,4′-diaminodiphenylether, 3,3′-diaminodiphenylether, 4,4′-diaminobenzanilide, 3,3′-diaminobenzanilide, 1,4-bis(4-aminophenyl)benzene, 1,3-bis(4-aminophenyl)benzene, 1,4-bis(4-aminophenoxy)benzene, 1,3bis(4-aminophenoxy)benzene, 1,3-bis(3-aminophenoxy)benzene, 1,2-bis(4-aminophenoxy)ethane, 1,3-bis(4-aminophenoxy)propane, 1,4-bis(4-aminophenoxy)butane, 1,5-bis(4-aminophenoxy)pentane, 1,6-bis(4-aminophenoxy)hexane, bis[4-(4-aminophenoxy)phenyl]ether, bis[4-(3-aminophenoxy)phenyl]ether, 4,4′-bis(4-aminophenoxy)biphenyl, 3,4′-bis(4-aminophenoxy)biphenyl, 3,3′-bis(4-aminophenoxy)biphenyl, bis(4-aminophenoxyphenyl)sulfone, bis(3-aminophenoxyphenyl)sulfone, bis[4-(4-aminophenoxy)phenyl]sulfone, bis[4-(3-aminophenoxy)phenyl]sulfone, bis[4-(4-aminophenoxy)phenyl]ketone, 2,2-bis[4-{4-amino-2-(trifluoromethyl)phenoxy}phenyl]hexafluoropropane, 9,9-bis(4-aminophenyl)fluorene, 9,9-bis(4-amino-3-methylphenyl)fluorene, 2,7-diaminofluorene, 2-(4-aminophenyl)-5-aminobenzooxazole, 2-(3-aminophenyl)-5-aminobenzooxazole, 2-(4-aminophenyl)-6-aminobenzooxazole, 2-(3-aminophenyl)-6-aminobenzooxazole, 1,4-bis(5-amino-2-benzooxazolyl)benzene, 1,4-bis(6-amino-2-benzooxazolyl)benzene, 1,3-bis(5-amino-2-benzooxazolyl)benzene, 1,3-bis(6-amino-2-benzooxazolyl)benzene, 2,6-bis(4-aminophenyl)benzobisoxazole, 2,6-bis(3-aminophenyl)benzobisoxazole, bis[(3-aminophenyl)-5-benzooxazolyl], bis[(4-aminophenyl)-5-benzooxazolyl], bis[(3-aminophenyl)-6-benzooxazolyl], bis[(4-aminophenyl)-6-benzooxazolyl], 3,4′-diaminodiphenylsulfide, 4,4′-diaminodiphenylsulfide, 4,4′-[1,4-phenylenebis(1-methylethane -1,1-diyl)]dianiline, 4-aminobenzoic acid 4-aminophenylester, 1,3-bis(4-anilino)tetramethyldisiloxane, 1,4-bis(3-aminopropyldimethylsilyl)benzene, ortho-tolidinesulfone, and the like.

Specific examples of the aromatic diol represented by Formula (A5-2) include hydroquinone, resorcinol, 2,4-dihydroxytoluene, 4,4′-dihydroxybiphenyl, 3,3′-dihydroxybiphenyl, 3,4′-dihydroxybiphenyl, 1,4-dihydroxynaphthalene, 1,5-dihydroxynaphthalene, 2,6-dihydroxynaphthalene, 2,7-dihydroxynaphthalene, 9,10-dihydroxyanthracene, 9,10-bis(4-hydroxyphenyl)anthracene, 4,4′-dihydroxy-2,2′-bis(trifluoromethyl)biphenyl, 4,4′-dihydroxybenzophenone, 3,3′-dihydroxybenzophenone, 3,4′-dihydroxybenzophenone, 4,4′-dihydroxydiphenylsulfone, 3,3′-dihydroxydiphenylsulfone, 3,4′-dihydroxydiphenylsulfone, 4,4′-dihydroxydiphenylsulfide, 3,3′-dihydroxydiphenylsulfide, 3,4′-dihydroxydiphenylsulfide, 4,4′-dihydroxydiphenylmethane, 3,3′-diaminodiphenylmethane, 3,4′-dihydroxydiphenylmethane, 2,2-bis(4-hydroxyphenyl)propane (bisphenol A), 2,2-bis[4-(4-hydroxyphenoxy)phenyl]propane, 2,2-bis[4-(4-hydroxyphenoxy)phenyl]hexafluoropropane, 4,4′-dihydroxydiphenylether, 3,4′-dihydroxydiphenylether, 3,3′-dihydroxydiphenylether, 4,4′-dihydroxybenzanilide, 3,3′-dihydroxybenzanilide, 1,4-bis(4-hydroxyphenyl)benzene, 1,3-bis(4-hydroxyphenyl)benzene, 1,4-bis(4-hydroxyphenoxy)benzene, 1,3-bis(4-hydroxyphenoxy)benzene, 1,3-bis(3-hydroxyphenoxy)benzene, 1,2-bis(4-hydroxyphenoxy)ethane, 1,3-bis(4-hydroxyphenoxy)propane, 1,4-bis(4-hydroxyphenoxy)butane, 1,5-bis(4-hydroxyphenoxy)pentane, 1,6-bis(4-hydroxyphenoxy)hexane, bis[4-(4-hydroxyphenoxy)phenyl]ether, bis[4-(3-hydroxyphenoxy)phenyl]ether, 4,4′-bis(4-hydroxyphenoxy)biphenyl, 3,4′-bis(4-hydroxyphenoxy)biphenyl, 3,3′-bis(4-hydroxyphenoxy)biphenyl, bis(4-hydroxyphenoxyphenyl)sulfone, bis(3-hydroxyphenoxyphenyl)sulfone, bis[4-(4-hydroxyphenoxy)phenyl]sulfone, bis[4-(3-hydroxyphenoxy)phenyl]sulfone, bis[4-(4-hydroxyphenoxy)phenyl]ketone, 2,2-bis[4-{4-hydroxy2-(trifluoromethyl)phenoxy}phenyl]hexafluoropropane, 9,9-bis(4-hydroxyphenyl)fluorene, 9,9-bis(4-hydroxy-3-methylphenyl)fluorene, 2,7-dihydroxyfluorene, 2-(4-hydroxyphenyl)-5-hydroxybenzooxazole, 2-(3-hydroxyphenyl)-5-hydroxybenzooxazole, 2-(4-hydroxyphenyl)-6-hydroxybenzooxazole, 2-(3-hydroxyphenyl)-6-hydroxybenzooxazole, 1,4-bis(5-hydroxy-2-benzooxazolyl)benzene, 1,4-bis(6-hydroxy-2-benzooxazolyl)benzene, 1,3-bis(5-hydroxy-2-benzooxazolyl)benzene, 1,3-bis(6-hydroxy-2-benzooxazolyl)benzene, 2,6-bis(4-hydroxyphenyl)benzobisoxazole, 2,6-bis(3-hydroxyphenyl)benzobisoxazole, bis[(3-hydroxyphenyl)-5benzooxazolyl], bis[(4-hydroxyphenyl)-5-benzooxazolyl], bis[(3-hydroxyphenyl)-6-benzooxazolyl], bis[(4-hydroxyphenyl) -6-benzooxazolyl], 3,4′-dihydroxydiphenylsulfide, 4,4′-dihydroxydiphenylsulfide, 4,4′-[1,4-phenylenebis(1-methylethane-1,1-diyl)]dianiline, 4-hydroxybenzoic acid 4-hydroxyphenylester, 1,3-bis(4-anilino)tetramethyldisiloxane, and the like.

Specific examples of the aromatic dithiol represented by Formula (A5-3) include 1,4-dimercaptobenzene, 1,3-dimercaptobenzene, 2,4-dimercaptotoluene, 4,4′-dimercaptobiphenyl, 3,3′-dimercaptobiphenyl, 3,4′-dimercaptobiphenyl, 1,4-dimercaptonaphthalene, 1,5-dimercaptonaphthalene, 2,6-dimercaptonaphthalene, 2,7-dimercaptonaphthalene, 9,10-dimercaptoanthracene, 9,10-bis(4-mercaptophenyl)anthracene, 4,4′-dimercapto-2,2′-bis(trifluoromethyl)biphenyl, 4,4′-dimercaptobenzophenone, 3,3′-dimercaptobenzophenone, 3,4′-dimercaptobenzophenone, 4,4′-dimercaptodiphenylsulfone, 3,3′-dimercaptodiphenylsulfone, 3,4′-dimercaptodiphenylsulfone, 4,4′-dimercaptodiphenylsulfide, 3,3′-dimercaptodiphenylsulfide, 3,4′-dimercaptodiphenylsulfide, 4,4′-dimercaptodiphenylmethane, 3,3′-diaminodiphenylmethane, 3,4′-dimercaptodiphenylmethane, 2,2-bis(4-mercaptophenyl)propane (bisphenol A), 2,2-bis[4-(4-mercaptophenoxy)phenyl]propane, 2,2-bis[4-(4-mercaptophenoxy)phenyl]hexafluoropropane, 4,4′-dimercaptodiphenylether, 3,4′-dimercaptodiphenylether, 3,3′-dimercaptodiphenylether, 4,4′-dimercaptobenzanilide, 3,3′-dimercaptobenzanilide, 1,4-bis(4-mercaptophenyl)benzene, 1,3-bis(4-mercaptophenyl)benzene, 1,4-bis(4-mercaptophenoxy)benzene, 1,3-bis(4-mercaptophenoxy)benzene, 1,3-bis(3-mercaptophenoxy)benzene, 1,2-bis(4-mercaptophenoxy)ethane, 1,3-bis(4-mercaptophenoxy)propane, 1,4-bis(4-mercaptophenoxy)butane, 1,5-bis(4-mercaptophenoxy)pentane, 1,6-bis(4-mercaptophenoxy)hexane, bis[4-(4-mercaptophenoxy)phenyl]ether, bis[4-(3-mercaptophenoxy)phenyl]ether, 4,4′-bis(4-mercaptophenoxy)biphenyl, 3,4′-bis(4-mercaptophenoxy)biphenyl, 3,3′-bis(4-mercaptophenoxy)biphenyl, bis(4-mercaptophenoxyphenyl)sulfone, bis(3-mercaptophenoxyphenyl)sulfone, bis[4-(4-mercaptophenoxy)phenyl]sulfone, bis[4-(3-mercaptophenoxy)phenyl]sulfone, bis[4-(4-mercaptophenoxy)phenyl]ketone, 2,2-bis[4-{4-mercapto2-(trifluoromethyl)phenoxy}phenyl]hexafluoropropane, 9,9-bis(4-mercaptophenyl)fluorene, 9,9-bis(4-mercapto3-methylphenyl)fluorene, 2,7-dimercaptofluorene, 2-(4-mercaptophenyl)-5-mercaptobenzooxazole, 2-(3-mercaptophenyl) -5-mercaptobenzooxazole, 2-(4-mercaptophenyl)-6-mercaptobenzooxazole, 2-(3-mercaptophenyl)-6-mercaptobenzooxazole, 1,4-bis(5-mercapto-2-benzooxazolyl)benzene, 1,4-bis(6-mercapto-2-benzooxazolyl)benzene, 1,3-bis(5-mercapto-2-benzooxazolyl)benzene, 1,3-bis(6-mercapto-2-benzooxazolyl)benzene, 2,6-bis(4-mercaptophenyl)benzobisoxazole, 2,6-bis(3-mercaptophenyl)benzobisoxazole, bis[(3-mercaptophenyl)-5-benzooxazolyl], bis[(4-mercaptophenyl)-5-benzooxazolyl], bis[(3-mercaptophenyl)-6-benzooxazolyl], bis[(4-mercaptophenyl)-6-benzooxazolyl], 3,4′-dimercaptodiphenylsulfide, 4,4′-dimercaptodiphenylsulfide, 4,4′-[1,4-phenylenebis(1-methylethane-1,1-diyl)]dianiline, 4-mercaptobenzoic acid 4-mercaptophenylester, 1,3-bis(4-anilino)tetramethyldisiloxane and the like.

The triazine compound represented by Formula (A3) can be produced by condensing cyanuric halide with an aromatic amine compound represented by Formula (A3-1) below, a hydroxyaromatic compound represented by Formula (A3-2) below, or a mercaptoaromatic compound represented by Formula (A3-3) below.

R^{a1}—Ar¹—NR^{a2}H (A3-1)

R^{a1}—Ar¹—OH (A3-2)

R^{a1}—Ar¹—SH (A3-3)

In Formulae (A3-1) to (A3-3), Ar¹, R^{a1}, and R^{a2} are the same as described for Formula (A1).

Examples of the cyanuric halide include cyanuric fluoride, cyanuric chloride, and cyanuric bromide. Among them, cyanuric chloride is preferred.

An amount of the aromatic amine compound represented by Formula (A3-1), the hydroxyaromatic compound represented by Formula (A3-2), or the mercaptoaromatic compound represented by Formula (A3-3) for use in production of the triazine compound represented by Formula (A3) is preferably 0.8 moles or more and 1.2 moles or less, more preferably 0.9 moles or more and 1.1 moles or less, and further preferably 0.95 moles or more and 1.05 moles or less relative to 1 mole of cyanuric halide.

The reaction of the cyanuric halide with the aromatic amine compound represented by Formula (A3-1), the hydroxyaromatic compound represented by Formula (A3-2), or the mercaptoaromatic compound represented by Formula (A3-3) is usually performed by mixing them in an organic solvent. The organic solvent for use in such a reaction is not particularly limited as long as the reaction proceeds well. The organic solvent is preferably a solvent having no hydroxy group, mercapto group, or amino group since it does not react with the cyanuric halide. Specific examples of the organic solvent include ethers such as tetrahydrofuran and 1,4-dioxane; ketones such as acetone, methyl ethyl ketone, methyl isopropyl ketone, diethyl ketone, methyl isobutyl ketone, methyl n-butyl ketone, and cyclohexanone; aromatic hydrocarbons such as toluene, p-xylene, o-xylene, m-xylene, ethylbenzene, and styrene; monoalkyl ether acetates of glycols such as ethylene glycol methyl ether acetate, ethylene glycol monoethyl ether acetate, propylene glycol monomethyl ether acetate, and diethylene glycol monoethyl ether acetate; dialkyl ethers of glycols such as ethylene glycol dimethyl ether, diethylene glycol dimethyl ether, diethylene glycol diethyl ether, and triethylene glycol dimethyl ether; esters such as ethyl acetate, n-propyl acetate, isopropyl acetate, n-butyl acetate, and isobutyl acetate; and lactones such as γ-butyrolactone. An amount of the organic solvent used is not particularly limited as long as the reaction proceeds well. The amount of the organic solvent used is preferably 100 parts by mass or more and 5000 parts by mass or less and more preferably 200 parts by mass or more and 4000 parts by mass or less relative to 100 parts by mass of reaction materials.

A temperature at which the cyanuric halide is reacted with the aromatic amine compound represented by Formula (A3-1), the hydroxyaromatic compound represented by Formula (A3-2), or the mercaptoaromatic compound represented by Formula (A3-3) is preferably −20° C. or more and 150° C. or less and more preferably −10° C. or more and 50° C. or less. When the aromatic amine compound represented by Formula (A3-1), the hydroxyaromatic compound represented by Formula (A3-2), or the mercaptoaromatic compound represented by Formula (A3-3) has a tert-butoxycarbonyloxy group or a tert-butoxycarbonylamino group as R^{a1}, the temperature is preferably −10° C. or more and 50° C. or less in order to prevent deprotection of the tert-butoxycarbonyloxy group or tert-butoxycarbonylamino group with heat. A reaction time is not particularly limited. For example, the reaction time is preferably 30 minutes or more and 50 hours or less and more preferably 1 hour or more and 30 hours or less.

Suitable specific examples of the aromatic amine compound represented by Formula (A3-1), the hydroxyaromatic compound represented by Formula (A3-2), and the mercaptoaromatic compound represented by Formula (A3-3) include the following compounds. R^{a1} in formulae below is the same as described for R^{a1} in Formula (A1).

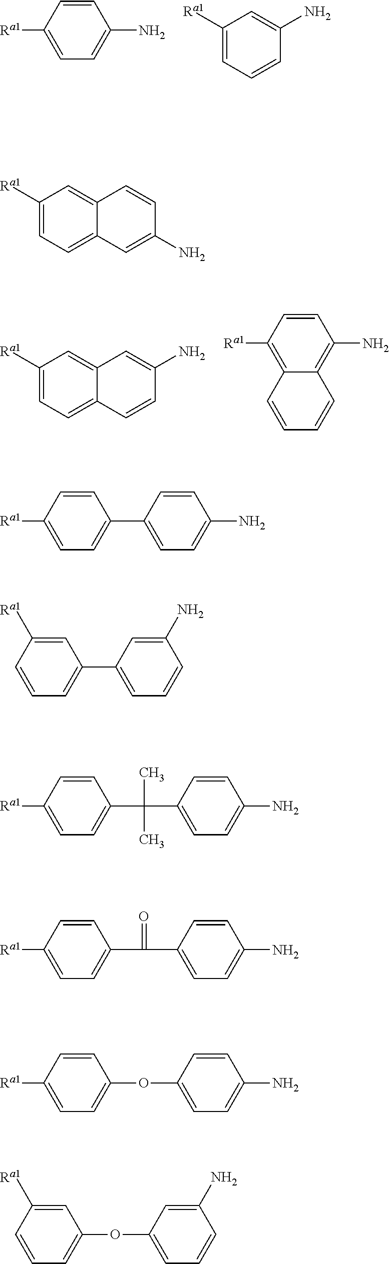

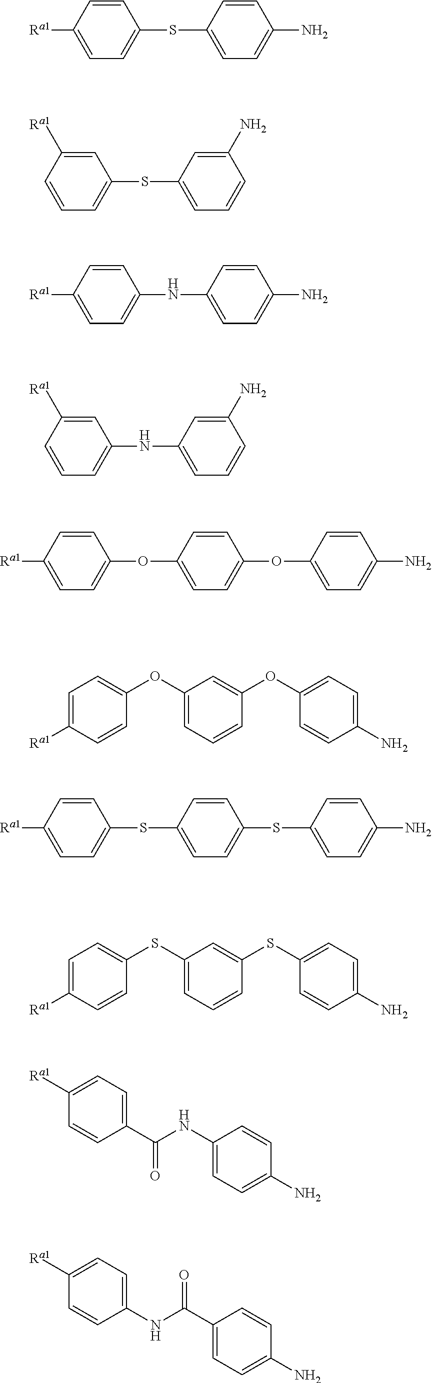

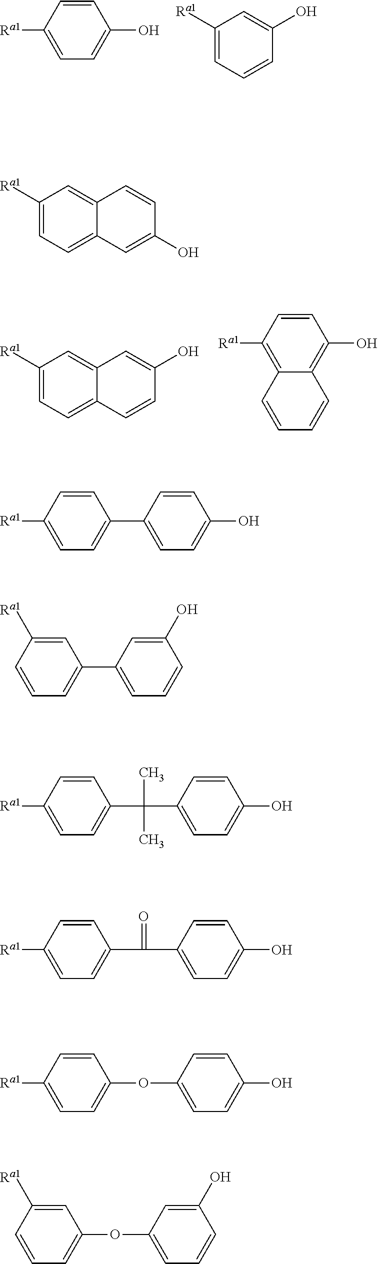

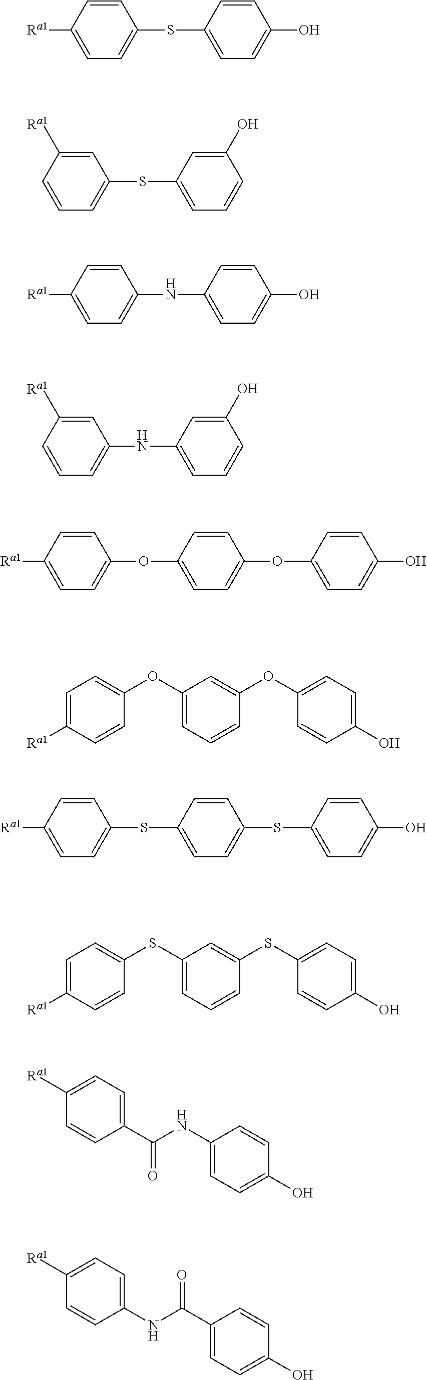

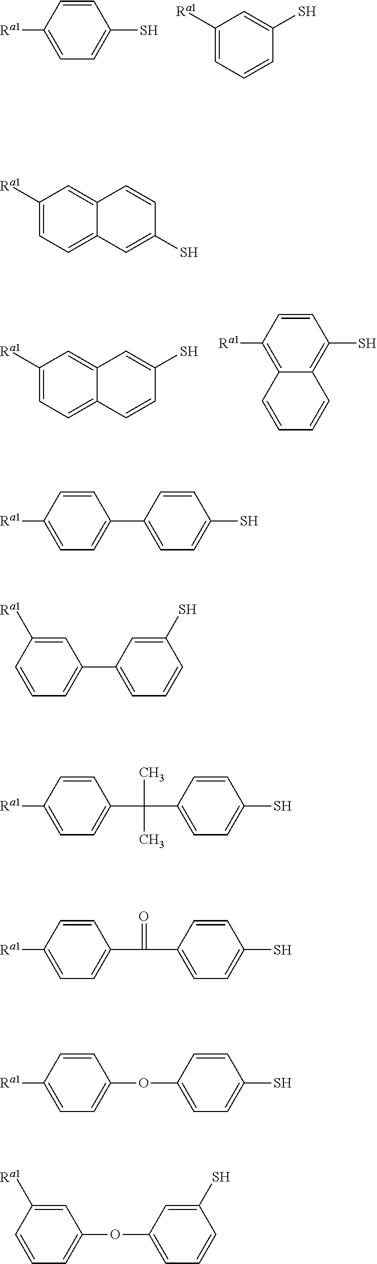

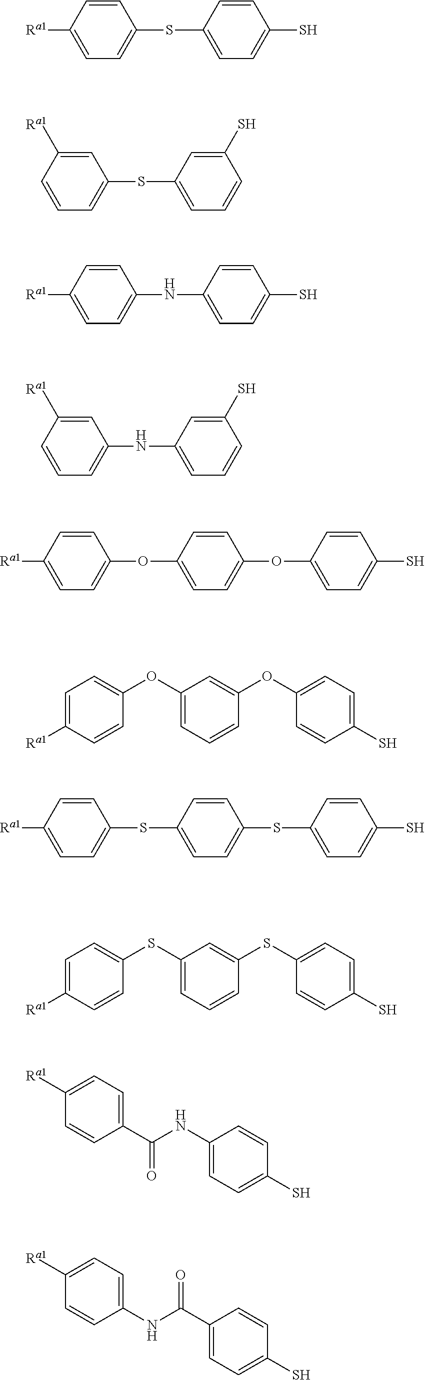

The unit (A1) and the unit (A2) can be obtained by using a dihalotriazine compound other than the triazine compound represented by Formula (A3) in addition to the triazine compound represented by Formula (A3). The dihalotriazine compound other than the triazine compound represented by Formula (A3) is preferably a dihalotriazine compound represented by Formula (A2-1) below. Use of the dihalotriazine compound represented by Formula (A2-1) allows a unit represented by Formula (A2) to be incorporated into the polymer as the unit (A2).

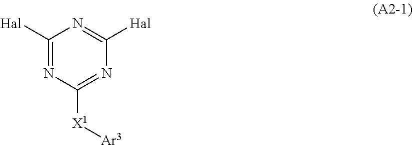

X¹ in Formula (A2-1) is the same as described for X¹ in Formula (A1). Ar³ in Formula (A2-1) is the same as described for Ar³ in Formula (A2). Hal in Formula (A2-1) is the same as described for Hal in Formula (A3).

A method for condensing a dihalotriazine compound including the triazine compound represented by Formula (A3) with the aromatic diamine compound represented by Formula (A5-1), the aromatic diol represented by Formula (A5-2), or the aromatic dithiol represented by Formula (A5-3) is not particularly limited. Typically, the above-described polymer can be produced by mixing the dihalotriazine compound including the triazine compound represented by Formula (A3) with the aromatic diamine compound represented by Formula (A5-1), the aromatic diol represented by Formula (A5-2), or the aromatic dithiol represented by Formula (A5-3) in an organic solvent.

The organic solvent for use in the above-mentioned condensation reaction is preferably an organic solvent in which the resulting polymer is soluble. Examples of such an organic solvent include nitrogen-containing polar organic solvents such as N,N-dimethylacetoamide, N,N-diethylacetoamide, N,N-dimethylformamide, N-methylformamide, N,N-dimethylpropionic amide, N,N-dimethylisobutylamide, N-methyl-2-pyrrolidone, N-ethyl-2-pyrrolidone, N-methyl-2-piperidone, N-acetylpyrrolidine, N,N′-dimethylethyleneurea (1,3-dimethyl-2-imidazolidinone), N,N′-dimethylpropyleneurea, N,N,N′,N′-tetramethylmalonic amide, N-methylcaprolactam, and N,N,N′,N′-tetramethylurea; sulfoxides such as dimethylsulfoxide; hexamethylphosphoric triamide; ketone solvents such as cyclopentanone, cyclohexanone, and methyl isobutyl ketone; and ethers such as tetrahydrofuran and dioxane. An amount of the organic solvent used is not particularly limited as long as the reaction proceeds well. The amount of the organic solvent used is preferably 100 parts by mass or more and 5000 parts by mass or less and more preferably 200 parts by mass or more and 4000 parts by mass or less relative to 100 parts by mass of reaction materials.

An amount of the aromatic diamine compound represented by Formula (A5-1), the aromatic diol represented by Formula (A5-2), or the aromatic dithiol represented by Formula (A5-3) for use in production of the polymer is preferably 0.8 moles or more and 1.2 moles or less, more preferably 0.9 moles or more and 1.1 moles or less, and further preferably 0.95 moles or more and 1.05 moles or less relative to 1 mole of the dihalotriazine compound including the triazine compound represented by Formula (A3).

A reaction temperature at which the polymer is produced is, for example, preferably 0° C. or more and 200° C. or less and more preferably 20° C. or more and 150° C. or less. A reaction time is not particularly limited. For example, the reaction time is preferably 5 minutes or more and 24 hours or less, more preferably 10 minutes or more and 18 hours or less, and further preferably 30 minutes or more and 12 hours or less.

### <<Non-Photosensitive Liquid Composition>>

A non-photosensitive liquid composition includes the above-described polymer(A) and an organic solvent (S). When the above-described polymer(A) has a hydroxy group protected with an acetal protecting group serving as R^{a1}, the non-photosensitive liquid composition further includes a thermal acid generating agent (C).

Such a non-photosensitive liquid composition is molded into a desired shape with a method such as coating or casting and then heated to remove the organic solvent (S). Thus, a molded product can be obtained. During this process, the hydroxy group protected with a tert-butoxycarbonyloxy group, a tert-butoxycarbonylamino group, or an acetal protecting group is deprotected with heating to thereby produce a hydroxy group or an amino group. Therefore, a polymer component included in the molded product is less soluble in the organic solvent. When the polymer(A) has a hydroxy group protected with an acetal protecting group serving as R^{a1}, the non-photosensitive liquid composition further includes a thermal acid generating agent (C). When such a non-photosensitive liquid composition is heated, the hydroxy group protected with an acetal protecting group is deprotected by the action of an acid that the thermal acid generating agent (C) generates to thereby produce a hydroxy group. Therefore, a polymer component included in the molded product is less soluble in the organic solvent.

### <Polymer (A)>

A polymer (A) is the above-described polymer including the unit represented by Formula (A1).

<Organic solvent (S)>

The organic solvent (S) is preferably a solvent in which the polymer(A) can be dissolved. Examples of the organic solvent (S) include nitrogen-containing polar organic solvents such as N,N-dimethylacetoamide, N,N-diethylacetoamide, N,N-dimethylformamide, N-methylformamide, N,N-dimethylpropionic amide, N,N-dimethylisobutylamide, N-methyl-2-pyrrolidone, N-ethyl-2-pyrrolidone, N-methyl-2-piperidone, N-acetylpyrrolidine, N,N′-dimethylethyleneurea (1,3-dimethyl-2-imidazolidinone), N,N′-dimethylpropyleneurea, N,N,N′,N′-tetramethylmalonic amide, N-methylcaprolactam, and N,N,N′,N′-tetramethylurea; ketones such as acetone, methyl ethyl ketone, methyl isopropyl ketone, diethyl ketone, methyl isobutyl ketone, methyl n-butyl ketone, cyclopentanone, and cyclohexanone; ethers such as tetrahydrofuran and 1,4-dioxane; aromatic hydrocarbons such as toluene, p-xylene, o-xylene, m-xylene, ethylbenzene, and styrene; monoalkyl ether acetates of glycols such as ethylene glycol methyl ether acetate, ethylene glycol monoethyl ether acetate, propylene glycol monomethyl ether acetate, and diethylene glycol monoethyl ether acetate; monoalkyl ethers of glycols such as ethylene glycol monomethyl ether, ethylene glycol monoethyl ether, ethylene glycol monoisopropyl ether, ethylene glycol monobutyl ether, propylene glycol monomethyl ether, propylene glycol monoethyl ether, propylene glycol monobutyl ether, diethylene glycol monomethyl ether, diethylene glycol monoethyl ether, dipropylene glycol monomethyl ether, dipropylene glycol monoethyl ether, and 1-methoxy-2-butanol; dialkyl ethers of glycols such as ethylene glycol dimethyl ether, diethylene glycol dimethyl ether, diethylene glycol diethylether, and triethylene glycol dimethyl ether; glycols such as ethylene glycol, propylene glycol, trimethylene glycol, 1,3-butanediol, 1,4-butanediol, 2,3-butanediol, hexylene glycol, and diethylene glycol; alcohols such as methanol, ethanol, n-propanol, isopropanol, n-butanol, isobutanol, tert-butanol, 1-pentanol, 2-methyl-1-butanol, 2-methyl-2-butanol, 2-methyl-1-pentanol, 1-octanol, 2-ethylhexanol, allyl alcohol, 1-methoxy -2-butanol, cyclohexanol, diacetone alcohol, furfuryl alcohol, tetrahydrofurfuryl alcohol, and benzyl alcohol; esters such as ethyl acetate, n-propyl acetate, isopropyl acetate, n-butyl acetate, and isobutyl acetate; and lactones such as γ-butyrolactone. The organic solvent (S) preferably includes a ketone solvent or a nitrogen-containing polar organic solvent from the viewpoint of particularly good solubility of the polymer(A).

An amount of the organic solvent (S) used is not particularly limited as long as the non-photosensitive liquid composition can be molded into a desired shape. The organic solvent (S) is used so as to give a solid content concentration of the non-photosensitive liquid composition of preferably 1% by mass or more and 50% by mass or less and more preferably 5% by mass or more and 40% by mass or less.

### <Thermal Acid Generating Agent (C)>

When the polymer(A) has the hydroxy group protected with an acetal protecting group serving as R^{a1}, the non-photosensitive liquid composition further includes a thermal acid generating agent (C). The thermal acid generating agent (C) is not particularly limited as long as it is a compound generating an acid upon heating. A known thermal acid generating agent may be appropriately selected as the thermal acid generating agent (C). The thermal acid generating agent (C) is preferably an onium salt thermal acid generating agent from the viewpoint of an acid generating ability upon heating. Examples of the onium salt include a sulfonium ion, an ammonium ion, an iodonium ion, and a phosphonium ion. These are preferred from the viewpoints of stability and easy handling. Among these onium salts, the sulfonium ion and the iodonium ion are more preferred and the sulfonium ion is more preferred.

An amount of the thermal acid generating agent (C) used is not particularly limited as long as a desired effect is not impaired. The amount of the thermal acid generating agent (C) used is preferably 0.1% by mass or more and 15% by mass or less and more preferably 0.5% by mass or more and 10% by mass or less relative to a mass of a solid content of the non-photosensitive liquid composition.

### <Surfactant (D)>

The non-photosensitive liquid composition may further contain a surfactant (surface modifying agent) in order to improve a film formation ability, coatability, an anti-foaming property, and a leveling property. The surfactant may be used alone or two or more may be used in combination. Examples of the surfactant include a silicone surfactant, a fluorosurfactant, and a polymeric wetting and dispersing agent, with the polymeric wetting and dispersing agent being preferred from the viewpoint of improvement of a film formation ability.

Specific examples of the silicone surfactant include BYK-077, BYK-085, BYK-300, BYK-301, BYK-302, BYK-306, BYK-307, BYK-310, BYK-320, BYK-322, BYK-323, BYK-325, BYK-330, BYK-331, BYK-333, BYK-335, BYK-341, BYK-344, BYK-345, BYK-346, BYK-348, BYK-354, BYK-355, BYK-356, BYK-358, BYK-361, BYK-370, BYK-371, BYK-375, BYK-380, and BYK-390 (manufactured by BYK Chemie).

Specific examples of the fluorosurfactant include F-114, F-177, F-410, F-411, F-450, F-493, F-494, F-443, F-444, F-445, F-446, F-470, F-471, F-472SF, F-474, F-475, F-477, F-478, F-479, F-480SF, F-482, F-483, F-484, F-486, F-487, F-172D, MCF-350SF, TF-1025SF, TF-1117SF, TF-1026SF, TF-1128, TF-1127, TF-1129, TF-1126, TF-1130, TF-1116SF, TF-1131, TF-1132, TF-1027SF, TF-1441, TF-1442 (manufacture by DIC Corporation); and Polyfox series PF-636, PF-6320, PF-656, PF-6520 (manufactured by OMNOVA Solutions Inc.).

Specific examples of the polymeric wetting and dispersing agent include BYK-140, BYK-145, BYK-161, BYK-162, BYK-163, BYK-164, BYK-167, BYK-168, BYK-170, BYK-171, BYK-174, BYK-180, BYK-182, BYK-184, BYK-185, BYK-2050, BYK-2055, BYK-2015, and BYK-9077 (manufactured by BYK Chemie).

An amount of the surfactant used is not particularly limited. From the viewpoints of a film formation ability, coatability, an anti-foaming property, and a leveling property of the non-photosensitive liquid composition, the amount of the surfactant used is preferably 0.01% by mass or more and 2% by mass or less and more preferably 0.05% by mass or more and 1% by mass or less relative to a mass of a solid content of the non-photosensitive liquid composition.

### <Other Components>

The non-photosensitive liquid composition may include an additive such as an anti-foaming agent, a silane coupling agent, a coloring agent (pigment, dye), and a cross-linking agent, as necessary. Conventionally known compounds may be used for the additives.

### <Method for Producing Non-Photosensitive Liquid Composition>

The non-photosensitive liquid composition can be produced by mixing the above-mentioned essential components with an optional component as necessary and then uniformly dissolving the components in the organic solvent (S). The non-photosensitive liquid composition may be filtered through a filter with a desired opening as necessary.

### <<Method for Producing Molded Product>>

A molded product can be produced by a method including molding the above-mentioned non-photosensitive liquid composition; and removing an organic solvent (S) with heating from the non-photosensitive liquid composition having been molded.

A method for molding is not particularly limited and appropriately selected depending on a shape of the molding product. The method for molding may be, for example, coating or casting. Furthermore, the non-photosensitive liquid composition may be molded into a desired 3D shape using a so-called 3D printing method. A method for producing a film will be described hereinafter as a representative example of the method for producing a molded product.

First, a desired substrate is coated with the non-photosensitive liquid composition to form a coating film.

A method for coating the substrate with the non-photosensitive liquid composition is not particularly limited. For example, a contact transfer coating device such as a roll coater, a reverse coater, a bar coater, and a slit coater; a spinner (rotating coating device); or a non-contact coating device such as a curtain flow coater can be used to coat a substrate with the non-photosensitive liquid composition to give a desired film thickness to form a coating film.

Then, the coating film is heated to remove an organic solvent (S) from the coating film. A heating temperature is appropriately determined in view of a boiling point of the organic solvent (S), a temperature at which the acid generating agent (C) generates an acid, and the like. A temperature at which the coating film is heated is preferably 100° C. or more and 400° C. or less and more preferably 150° C. or more and 300° C. or less. A heating time is preferably 30 seconds or more and 30 minutes or less and more preferably 1 minute or more and 20 minutes or less.

The thus-produced molded product exhibits a high refractive index. Specifically, the molded product preferably has a refractive index of 1.71 or more and more preferably 1.75 or more as a refractive index for light having a wavelength of 550 nm. A molded product exhibiting such a high refractive index is preferably a film or microlens. The molded product as a film can be used as a high-refractive film in various optical element. The molded product as a microlens can be particularly suitably used in an optical element such as CCD and CMOS.

As mentioned above, the present inventors provide the following aspects (1) to (15).
(1) A polymer including
   a unit represented by Formula (A1) below:

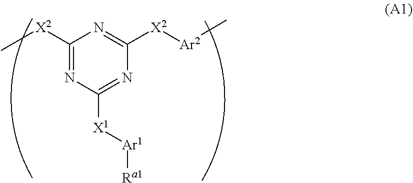

in which Ar¹ and Ar² are an aromatic group-containing group, R^{a1} is a hydroxy group protected with a tert-butoxycarbonyloxy group, a tert-butoxycarbonylamino group, or an acetal protecting group, the acetal protecting group being a group represented by —CHR^{A1}—O—R^{A2}, R^{A1} is a hydrogen atom or an alkyl group, R^{A2} is an alkyl group, R^{A1} and R^{A2} may be linked to each other to form a ring, X¹ and X² are each independently —NR^{a2}—, —O—, or —S—, R^{A2} is a hydrogen atom, an optionally substituted alkyl group, or an optionally substituted aromatic hydrocarbon group, X¹ is linked to an aromatic ring in the aromatic group-containing group serving as Ar¹, and X² is linked to an aromatic ring in the aromatic group-containing group serving as Ar².
(2) The polymer according to (1), in which R^{a1} is a tert-butoxycarbonyloxy group.
(3) The polymer according to (1) or (2), in which X² is —NR^{a2}— or —O—.
(4) A triazine compound represented by Formula (A3) below:

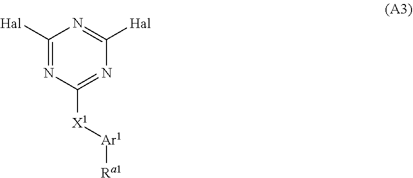

in which Hal is a halogen atom, Ar¹ is an aromatic group-containing group, R^{a1} is a hydroxy group protected with a tert-butoxycarbonyloxy group, a tert-butoxycarbonylamino group, or an acetal protecting group, the acetal protecting group being a group represented by —CHR^{A1}—O—R^{A2}, R^{A1} is a hydrogen atom or an alkyl group, R^{A2} is an alkyl group, R^{A1} and R^{A2} may be linked to each other to form a ring, X¹ is —NR^{a2}—, —O—, or —S—, R^{a2} is a hydrogen atom, an optionally substituted alkyl group, or an optionally substituted aromatic hydrocarbon group, and X¹ is linked to an aromatic ring in the aromatic group-containing group serving as Ar¹.
(5) A method for producing the triazine compound according to
(4), the method including: condensing cyanuric halide with a compound represented by any of Formulae (A3-1) to (A3-3) below:

R^{a1}—Ar¹—NR^{a2}H (A3-1)

R^{a1}—Ar¹—OH (A3-2)

R^{a1}—Ar¹—SH (A3-3)

in which Ar¹ is an aromatic group-containing group, R^{a1} is a hydroxy group protected with a tert-butoxycarbonyloxy group, a tert-butoxycarbonylamino group, or an acetal protecting group, and R^{a2} is a hydrogen atom, an optionally substituted alkyl group, or an optionally substituted aromatic hydrocarbon group.
(6) A method for producing a polymer, the method including: condensing the triazine compound according to (4) with a compound represented by any of Formulae (A5-1) to (A5-3) below:

Ar²—(NR^{a2}H)₂ (A5-1)

Ar²—(OH)₂ (A5-2)

Ar²—(SH)₂ (A5-3)

in which Ar² is an aromatic group-containing group, and R^{a2} is a hydrogen atom, an optionally substituted alkyl group, or an optionally substituted aromatic hydrocarbon group.
(7) A non-photosensitive liquid composition including: the polymer(A) according to any one of (1) to (3); and an organic solvent (S), and
   further including a thermal acid generating agent (C) when R^{a1} is a hydroxy group protected with an acetal protecting group.
(8) The non-photosensitive liquid composition according to (7), in which the organic solvent (S) is a ketone solvent or a nitrogen-containing polar organic solvent.
(9) A method for producing a molded product, the method including:
   molding the non-photosensitive liquid composition according to (7) or (8); and
   removing an organic solvent (S) with heating from the non-photosensitive liquid composition having been molded.
(10) The method for producing the molded product according to (9), in which the non-photosensitive liquid composition is molded into a film shape by coating a substrate with the non-photosensitive liquid composition.
(11) A molded product of the non-photosensitive liquid composition according to (7) or (8).
(12) The molded product according to (11) having a refractive index for light having a wavelength of 550 nm of 1.71 or more.
(13) A film formed of the molded product according to (11) or (12).
(14) A microlens formed of the molded product according to (11) or (12).
(15) An optical element including
   the microlens according to (14).

### EXAMPLES

The present invention will be described in more detail with reference to Examples and Comparative Examples. The present invention is not limited to the following Examples.

### Example 1

A 500 mL three-neck flask was charged with p-aminophenol (16.4 g, 150 mmol) and di-tert-butyl dicarbonate (34.4 g, 158 mmol), and 300 mL of tetrahydrofuran (THF) to thereby obtain a solution. Then, 4-N,N-dimethylaminopyridine (DMAP, 3.66 g, 30 mmol) was added portionwise to the flask. After the addition of DMAP, the resulting solution in the flask was stirred at room temperature overnight. After stirring overnight, THF was distilled off from the resulting reaction liquid to thereby obtain a solid residue. The resulting residue was purified by silica gel column chromatography to thereby obtain 24.4 g of 4-tert-butoxycarbonyloxyaniline.

A 500 mL three-neck flask was charged with cyanuric chloride (4.41 g, 24 mmol) dissolved in 85 mL of THF and the liquid in the flask was cooled to 0° C. in an ice bath. Then, 4-tert-butoxycarbonyloxyaniline (5.00 g, 24 mmol) dissolved in 85 mL of THF was added dropwise to the flask with the liquid in the flask being stirred. After the addition, stirring of the resulting reaction liquid in the flask was continued for 2 hours. The reaction liquid was transferred to a separating funnel, and then the separating funnel was charged with a potassium carbonate aqueous solution in which potassium carbonate (3.34 g, 24 mmol) was dissolved in 50 mL of pure water. An organic phase was washed with a potassium carbonate aqueous solution within the separating funnel and then an aqueous phase was removed via liquid separation. THF was removed from the thus-collected organic phase to thereby obtain a solid residue. The resulting residue was purified by silica gel column chromatography to thereby obtain 5.9 g of 2,4-dichloro-6-(4-tert-butoxycarbonyloxyphenyl)amino-1,3,5-triazine that is a dihalotriazine compound having the below-described structure. The ¹H NMR measurement result of the resulting compound is described below.

¹H NMR (400 MHz, CDCl₃): δ 7.98 (brs, 1H), 7.47 (d, JHH=8.8 Hz, 2H), 7.16 (d, JHH=8.8 Hz, 2H), 1.58 (s, 9H).

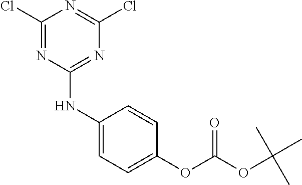 [SMILES: CC(C)(C)OC(=O)Oc1ccc(Nc2nc(Cl)nc(Cl)n2)cc1]

### Example 2

A 500 mL of three-neck flask was charged with 1,3-bis(4-aminophenoxy)benzene (4.39 g, 15 mmol) and 35 mL of N,N-dimethylacetoamide (DMAc) to thereby obtain a solution. The resulting solution in the flask was heated to 100° C. in an oil bath. Then, the flask was charged with a solution in which the dihalotriazine compound obtained in Example 1 (5.35 g, 15 mmol) was dissolved in 55 mL of DMAc to thereby initiate polymerization. After the reaction was performed for 3 hours since the initiation of polymerization, the resulting reaction liquid was cooled to room temperature. Four grams of an ammonia aqueous solution with a concentration of 28% by mass, 270 mL of pure water, and 100 mL of methanol were added to the flask to thereby precipitate a polymer produced from the reaction liquid in the flask. The resulting crude polymer was collected by filtering and then washed with 50 mL of hexane. The thus-washed polymer was dried at 100° C. for 10 hours in a vacuum drier to thereby obtain a polymer A-1 that is composed of a unit described below. Then, 1.0 g of the resulting polymer A-1 was mixed with 9 g of cyclopentanone, N-methyl-2-pyrrolidone, or propylene glycol monomethyl ether. The resulting mixed liquid was sonicated for 30 minutes and then verified for a solubility of the polymer A-1 in various solvents. After the sonication, the polymer A-1 was uniformly dissolved in any of the solvents.

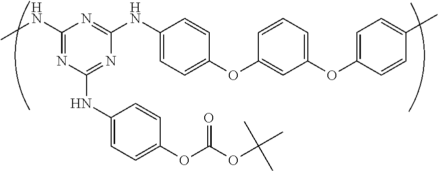

### Example 3

A polymer A-2 composed of a unit described below was obtained in the same manner as in Example 2, except that 1,3-bis(4-aminophenoxy)benzene (15 mmol) was changed to bis(4-aminophenyl)sulfide (15 mmol). Then, 1.0 g of the resulting polymer A-2 was mixed with 9 g of cyclopentanone, N-methyl-2-pyrrolidone, or propylene glycol monomethyl ether. The resulting mixed liquid was sonicated for 30 minutes and then verified for a solubility of the polymer A-2 in various solvents. After the sonication, the polymer A-2 was uniformly dissolved in cyclopentanone or N-methyl-2-pyrrolidone. The polymer A-2 remained partially undissolved in propylene glycol monomethyl ether.

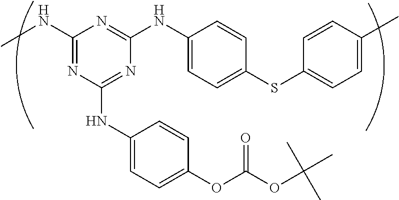

### Example 4

A polymer A-3 composed of a unit described below was obtained in the same manner as in Example 2, except that 1,3-bis(4-aminophenoxy)benzene (15 mmol) was changed to bis(4-mercaptophenyl)sulfide (15 mmol). Then, 1.0 g of the resulting polymer A-3 was mixed with 9 g of cyclopentanone, N-methyl-2-pyrrolidone, or propylene glycol monomethyl ether. The resulting mixed liquid was sonicated for 30 minutes and then verified for a solubility of the polymer A-3 in various solvents. After the sonication, the polymer A-3 was uniformly dissolved in any of the solvents.

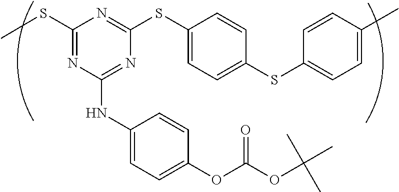

### Example 5

A 300 mL three-neck flask was charged with m-nitrophenol (9.74 g, 70 mmol), di-tert-butyl dicarbonate (16.0 g, 74 mmol), and 140 mL of tetrahydrofuran (THF) to thereby obtain a solution. Then, 4-N,N-dimethylaminopyridine (DMAP, 1.71 g, 14 mmol) was added to the flask. After the addition of DMAP, the resulting solution in the flask was stirred at room temperature overnight. After stirring overnight, THF was distilled off from the resulting reaction liquid to thereby obtain a solid residue. The resulting residue was purified by silica gel column chromatography to thereby obtain 16.2 g of 3-tert-butoxycarbonyloxynitrophenol.

A 300 mL three-neck flask was charged with 3-tert -butoxycarbonyloxynitrophenol (16.2 g, 68 mmol), ammonium formate (21.3 g, 339 mmol), and 330 mL of methanol to thereby obtain a solution. Then, palladium/carbon (5% by weight of palladium, 0.72 g, 0.339 mmol (in terms of palladium)) was added to the flask. After the addition of palladium/carbon, the resulting solution in the flask was stirred at room temperature overnight. After stirring overnight, the resulting reaction liquid was filtered with Celite. Methanol was distilled off from the resulting filtrate to thereby obtain a solid residue. The resulting residue was purified by silica gel column chromatography to thereby obtain 13.1 g of 3-tert-butoxycarbonyloxyaniline.

A 500 mL three-neck flask was charged with cyanuric chloride (11.5 g, 63 mmol) dissolved in 90 mL of THF and the liquid in the flask was cooled to 0° C. in an ice bath. Then, 3-tert-butoxycarbonyloxyaniline (13.1 g, 63 mmol) dissolved in 90 mL of THF was added dropwise to the flask with the liquid in the flask being stirred. After the addition, stirring of the resulting reaction liquid in the flask was continued for 2 hours. A potassium carbonate aqueous solution in which potassium carbonate (5.19 g, 38 mmol) was dissolved in 60 mL of pure water was added to the reaction liquid and stirred at room temperature for 20 minutes. The resulting reaction mixed liquid and 400 mL of ethyl acetate were transferred to a separating funnel and then an aqueous phase was removed via liquid separation. THF and ethyl acetate were removed from the thus-collected organic phase to thereby obtain a solid residue. The resulting residue was purified by silica gel column chromatography to thereby obtain 19.3 g of 2,4-dichloro-6-(3-tert-butoxycarbonyloxyphenyl)amino-1,3,5-triazine that is a dihalotriazine compound having the below-described structure. The ¹H NMR measurement result of the resulting compound is described below.

¹H NMR (400 MHz, CDCl₃): δ 7.90 (brs, 1H), 7.50 (dd, JHH=2.4, 2.0 Hz, 1H), 7.35-7.27 (m, 2H), 7.02-6.99 (m, 1H), 1.59 (s, 9H)

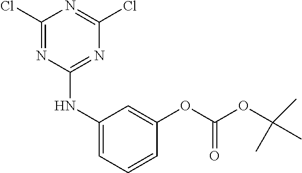 [SMILES: CC(C)(C)OC(=O)Oc1cccc(Nc2nc(Cl)nc(Cl)n2)c1]

### Example 6

A polymer A-4 composed of a unit described below was obtained in the same manner as in Example 2, except that 2,4-dichloro-6-(4-tert-butoxycarbonyloxyphenyl)amino-1,3,5-triazine (15 mmol) was changed to 2,4-dichloro-6-(3-tert -butoxycarbonyloxyphenyl)amino-1,3,5-triazine 4,4′-(m -phenylenedioxy)dianiline (15 mmol) and 1,3-bis(4-aminophenoxy)benzene (15 mmol) was changed to bis(4-aminophenylsulfide (15 mmol). Then, 1.0 g of the resulting polymer A-4 was mixed with 9 g of cyclopentanone, N-methyl-2-pyrrolidone, or propylene glycol monomethyl ether. The resulting mixed liquid was sonicated for 30 minutes and then verified for a solubility of the polymer A-4 in various solvents. After the sonication, the polymer A-4 was uniformly dissolved in cyclopentanone or N-methyl-2-pyrrolidone. The polymer A-4 remained partially undissolved in propylene glycol monomethyl ether.

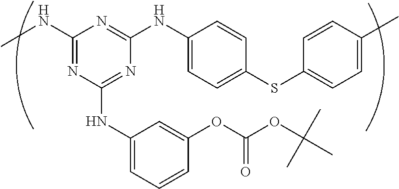

### Example 7

A 200 mL three-neck flask was charged with cyanuric chloride (3.69 g, 20 mmol) dissolved in 30 mL of THF and the liquid in the flask was cooled to 0° C. in an ice bath. Then, 4-tert-butoxy carbonylaminoaniline (4.17 g, 20 mmol) dissolved in 90 mL of THF was added dropwise to the flask with the liquid in the flask being stirred. After the addition, stirring of the resulting reaction liquid in the flask was continued for 2 hours. A potassium carbonate aqueous solution in which potassium carbonate (1.66 g, 12 mmol) was dissolved in 30 mL of pure water was added to the reaction liquid and stirred at room temperature for 20 minutes. The resulting reaction mixed liquid and 150 mL of ethyl acetate were transferred to a separating funnel and then an aqueous phase was removed via liquid separation. THF and ethyl acetate were removed from the thus-collected organic phase to thereby obtain a solid residue. The resulting residue was purified by silica gel column chromatography to thereby obtain 6.54 g of 2,4-dichloro-6-(4-tert-butoxy carbonylaminophenyl)amino-1,3,5-triazine that is a dihalotriazine compound having the below-described structure. The ¹H NMR measurement result of the resulting compound is described below.

¹H NMR (400 MHz, CDCl₃): δ 7.85 (brs, 1H), 7.44 (d, JHH=9.2 Hz, 2H), 7.38 (d, JHH=9.2 Hz, 2H), 6.63 (brs, 1H), 1.53 (s, 9H).

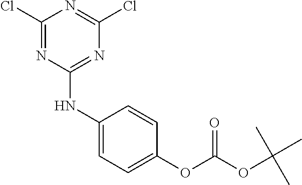 [SMILES: CC(C)(C)OC(=O)Oc1ccc(Nc2nc(Cl)nc(Cl)n2)cc1]

### Comparative Example 1

A polymer A-5 composed of a unit described below was obtained in the same manner as in Example 2, except that the dihalotriazine compound obtained in Example 1 (15 mmol) was changed to 2,4-dichloro-6-phenylamino-1,3,5-triazine (15 mmol).

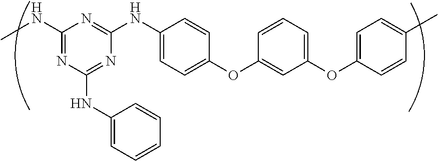

### Example 8, Example 9, Example 10, Example 11, and Comparative Example 2

Five grams of a polymer described in Table 1 and 0.003 g of a fluorosurfactant (PF-656, manufactured by OMNOVA Solutions Inc.) were dissolved in 45 g of cyclopentanone to thereby obtain a solution. The resulting solution was filtered through a filter with an opening of 0.45 μm. Thus, non-photosensitive liquid compositions of Example 8, Example 9, Example 10, Example 11, and Comparative Example 2 were obtained. The resulting non-photosensitive liquid compositions were measured for light transmittance according to the below-mentioned method. The measurement results are shown in Table 1. The resulting non-photosensitive liquid compositions were used to form films according to the below-mentioned method. According to the below-mentioned methods, the resulting films were measured for light transmittance, a refractive index, and a residual film rate after dipping in acetone. These measurement results are shown in Table 1.

### <Measurement of Light Transmittance>

A film formed of any of the non-photosensitive liquid compositions on a glass substrate was measured for light transmittance at a wavelength of 400 nm using an ultraviolet and visible spectrophotometer.

### <Film Formation>

A glass substrate and a silicon substrate were coated with any of the non-photosensitive liquid compositions using a spin coater. The-thus formed coating film was heated at 100° C. for 1 minute and then at 250° C. for 5 minutes to thereby obtain a film.

### <Measurement of Refractive Index>

The film on the silicon substrate was measured for a refractive index at a wavelength of 550 nm using a spectroscopic ellipsometer.

### <Measurement of Residual Film Rate>

The film on the silicon substrate was dipped in acetone at room temperature for 10 minutes. A film thickness before dipping T1 and a film thickness after dipping T2 were measured. A residual film rate was calculated from the expression below:

Residue film rate (%)=T2/T1*100

TABLE 1

| | | Light | Refractive | Residual film |
|---|---|---|---|---|
| | Polymer | transmittance | index | rate |
| Ex. 8 | A-1 | 99.0% | 1.72 | 66.4% |
| Ex. 9 | A-2 | 99.3% | 1.77 | 57.3% |
| Ex. 10 | A-3 | 94.7% | 1.76 | 87.2% |
| Ex. 11 | A-4 | 97.6% | 1.81 | 100.0% |
| Comp. Ex. 2 | A-5 | 99.0% | 1.72 | 9.0% |

It can be seen from Example 8, Example 9, Example 10, and Example 11 that use of a polymer including the unit (A1) represented by the above-described Formula (A1) enables formation of a molded product having both of a high refractive index and a high organic solvent resistance. On the other hand, it can be seen from Comparative Example 2 that use of a polymer composed of a unit not corresponding to the above-described Formula (A1) makes it difficult to form a molded product having an excellent organic solvent resistance.

What is claimed is:

1. A polymer comprising a unit represented by Formula (A1) below: 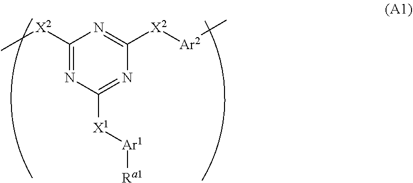
wherein Ar¹ and Ar² are an aromatic group-containing group, R^{a1} is a hydroxy group protected with a tert-butoxycarbonyloxy group, a tert-butoxycarbonylamino group, or an acetal protecting group, the acetal protecting group being a group represented by —CHR^{A1}—O—R^{A2}, R^{A1} is a hydrogen atom or an alkyl group, R^{A2} is an alkyl group, RA' and R^{A2} may be linked to each other to form a ring, X¹ and X² are each independently —NR^{a2}—, —O—, or —S—, R^{a2} is a hydrogen atom, an optionally substituted alkyl group, or an optionally substituted aromatic hydrocarbon group, X¹ is linked to an aromatic ring in the aromatic group-containing group serving as Ar¹, and X² is linked to an aromatic ring in the aromatic group-containing group serving as Ar².

2. The polymer according to claim 1, wherein R^{a1} is a tert-butoxycarbonyloxy group.

3. The polymer according to claim 1, wherein X² is —NR^{a2}—, or —O—.

4. A triazine compound represented by Formula (A3) below: 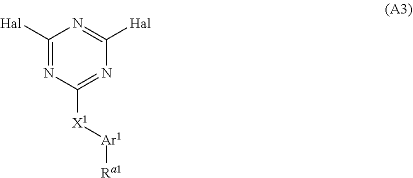
wherein Hal is a halogen atom, Ar¹ is an aromatic group-containing group, R^{a1} is a hydroxy group protected with a tert-butoxycarbonyloxy group, a tert-butoxycarbonylamino group, or an acetal protecting group, the acetal protecting group being a group represented by —CHR^{A1}—O—R^{A2}, R^{A1} is a hydrogen atom or an alkyl group, R^{A2} is an alkyl group, R^{A1} and R^{A2} may be linked to each other to form a ring, X¹ is —NR^{a2}—, —O—, or —S—, R^{a2} is a hydrogen atom, an optionally substituted alkyl group, or an optionally substituted aromatic hydrocarbon group, and X¹ is linked to an aromatic ring in the aromatic group-containing group serving as Ar¹.

5. A method for producing the triazine compound according to claim 4, the method comprising condensing cyanuric halide with a compound represented by any of Formulae (A3-1) to (A3-3) below:
R^{a1}—Ar¹—NR^{a2}H (A3-1)
R^{a1}—Ar¹—OH (A3-2)
R^{a1}—Ar¹—SH (A3-3)
wherein Ar¹ is an aromatic group-containing group, R^{a1} is a hydroxy group protected with a tert-butoxycarbonyloxy group, a tert-butoxycarbonylamino group, or an acetal protecting group, and R^{a2} is a hydrogen atom, an optionally substituted alkyl group, or an optionally substituted aromatic hydrocarbon group.

6. A method for producing a polymer, the method comprising condensing the triazine compound according to claim 4 with a compound represented by any of Formulae (A5-1) to
Ar²—(NR^{a2}H)₂ (A5-1)
Ar²—(OH)₂ (A5-2)
Ar²—(SH)₂ (A5-3)
wherein Ar² is an aromatic group-containing group, and R^{a2} is a hydrogen atom, an optionally substituted alkyl group, or an optionally substituted aromatic hydrocarbon group.

7. A non-photosensitive liquid composition comprising: the polymer(A) according to claim 1 and an organic solvent (S), further comprising a thermal acid generating agent (C) when R^{a1} is a hydroxy group protected with an acetal protecting group.

8. The non-photosensitive liquid composition according to claim 7, wherein the organic solvent (S) is a ketone solvent or a nitrogen-containing polar organic solvent.

9. A method for producing a molded product, the method comprising:
molding the non-photosensitive liquid composition according to claim 7; and
removing the organic solvent (S) with heating from the non-photosensitive liquid composition after the molding.

10. The method for producing a molded product according to claim 9, wherein the non-photosensitive liquid composition is molded into a film shape by coating a substrate with the non-photosensitive liquid composition.

11. A molded product of the non-photosensitive liquid composition according to claim 7.

12. The molded product according to claim 11 having a refractive index for light having a wavelength of 550 nm of 1.71 or more.

13. A film formed from the molded product according to claim 11.

14. A microlens formed from the molded product according to claim 11.

15. An optical element comprising the microlens according to claim 14.
